SEQUENCE LISTING (1 sequence)

SEQ ID NO: 1 - DNA, 26220 bp, Mus musculus
  Mus musculus strain C57BL/6J chromosome 11, GRCm38.p6 C57BL/6J
acaaaggtggaaaggagatttagtggagtggcctcagtggcctcagtcacagagcagaatggaggggaggtctgatcaaggcaggaggaccagatgctaggccaaagagtttaagtgcacctgttgaaagccggtagacatctttgagcaagagactgccgtgactgaaaggactatgtctgaaaattgatctgaccaggagcacagctggcctgaaagaggagcaggaagactactaagttcctagatgtccagaaccgatgcagaagtcttgaagcaggcatgctgtttgtggttagaagaatagcaaggcaagacctagaaattcagagcctctgtggctggtgagatagcaagcgttcaattctcagcaaccacatggtggctcacaaccatctgtaatgggatccgataccctctctggtatgtgtgaagacagctacagcgtacttgtatacataaaataaataagtaattcttttaaaaagaaagtaaaaagaaattagccccatttgcgtcattccagaataattcagtaattaagctcatatgggaaaaaatgtttgtggtgctagaaaactgaacccaaagttatctcaaaattttattttgggggctggagaggtggctcaggggttaagagcactgactgctcttctgaaggtcctgagttcagatcccagcaaccacatggtggctcacaaccatccacccccccagcccagaagtgtgtttgtttgtttgtttgttgactcactttcaccctgtaatacaattcaggctggcttggattctgctatatagcccaggctgatcttgaacaagtctcagcctatctgaatgctgggattacagatgtgagccaacacaccgagcaggtacaacgttggagttttagaaacaactgctgggatagcagtgcacacctttaatctcagcactctagaggcagaggcaggcaggggatctctgtgagctccaagccagccagggcaacatagtgagaacctgccttgaaaaataaagaaaaaacggcatgcaaatgaaagcttcaagacaagttttcgcctctttggcaagaagtaggtgcctgtctctcttccttgcatttgaacaggctccagctgtccaagagaattgagctttggggaggtaagatattaagctaaagccacagcccacgcctacgtagctggaaggagaactaatgaccaatttcaaccggaagcttgacatccagcaggtggccatctccagcaggcaccttaaaaattcagctctcacgctgtacaaaggtgtttcaataaacgagataaaaataactctgtgaaaatgccttgcagtgtgtgtggtaagcatcattagaacttacctatttaaagatttttcttcgctagggtcacagtcaagatacctaatgggtttgaatgctccttgatttaaaaacaattgtttttccacgaaaacgctaaagtgtattacggtcaagttaatctgtttcgagaagatttgtcaaggaaatcattcactttggccattaacaaaaacaggtggaaagaaagcaagcgaacagctggccctggaaatagaaagtgcgctctatctggagggccacattttaggagactcggggagcctgtagcacttggggttcacggtgtaccgcctggagagccagcgagggtgaactgaaaagcggacgatgaccgacacaccgctctgcctggaccccgcggggaccgagggaggggacccctggactagacaaaggctgcagccccgggaggccgggtgcgcgtgcgccacggaatgcgcgtgcgcgcggcggtcagcaccgcggcccgaggcgtggcgggacccggggagggaagcgcggagaggggcgagccgagcggcgcggagagaggagggttgaggggcgctcgcgtgcgccccctagcggtcggaaggggaggtgcccgcccaggcctcgctgcccgcccctcccctagggaggttcgcgctcccgtggctcgcgcctgcgctgcccgccggcctccagaacgcgctttctcggccgcgcgcgctctcagtctccgccacccaccagctccagcaccagcagcagcgccgccgccaccgcccaccttctgccgccgccgccacaaccaccttctcctccgctgtcctcttctgtcctcgccttctgtcgattatcaggtaagcgccgcagctctgaaatccagtcccctctggccctgtgcgccgtcctcctgccacccctcgccccagagatggatttagggagctgggcatcaatcaggcgcaacggcgggggcctctctgcgccctaggcgctggggagctgggaaggaacgcgtgcaagagatgcacgcacgcgagcgaatgcgtgtatctatgagtgctggggtccccactatcggctccgcgcaaatgaggggggctgtgcccgcagggtggggcgcggatgacggtgacctagggaaggggttgttctgtaatctggatctcagccttttgccgcaatgggcgtgggtctgtgagtgtgcatgggggtccaacgacgacccccgaaacagaacctgaaatcggatttctctgctcctgattgccatcagccctaaggaaggtgtggatcgggttctagaaaagatgactgccttcatgtctctccacctccctgacagcgattctgacacggccttcactggccctgatttaaggcggtggcccgccccctccccttttcctccttcagaagccggcaggggacatttgggggctgggaggaatccggaggatggggaggggtccaggcgctgtcacgttagtcgcccttccccctgcgcgcgcttggtacagtgacaggggcagcgccgtgcccgagagaaggtagtgcgcggatcccaatgtgggagctcgaagagtcaggggtcacctggcctcgcggtgtcagcgggctggctggcagcggctccccgctcgagggcgcgcagcgttgcagccaggcacagacccacgcgagggctgcaggtgcatttcggggcgcgtttctttctctgcgttctgcgccgggctcccggcatgtgcgctccctgccggaggcgcgggctggcgcgcagggctcgcccctcactgcggcagtgggtgtggacctgggtgggcgaggaggggggagtacgatgtgtctcctcccgctgccacccctctccgttttttcccccctctctgaacgcgaggtgccatctttttgcggcgagtcacgtctctatcgtagcatgccaagagctgtggccaggctttggaggacccggcttggacccatgttaaagaacagggggtgatgaacacggaagccttggcgttttgcctctgcagcggtgcgcaccagagcgttcacctgaggtcgtctaggggagctcgctgctttgttcgtcgtgtgatttaggatctgcgctcccagccctggttttttgggggagtgtgcggggggaggcgggaagctttcattcgacgaggatatttgcctacgcactcggagcagctccatccttttttcgccactcttttcagaccctttgttggatctacagacttgcggcagcccactccccagttcgacccagagccacatgggtgggattcgagatagactctcgtgtgcaggagccctcagaggaagcacatccctccccacacccaggccatcctgaggaccacaccccacttgtgtttcactgctaggggagccagataaccctcgggaatggggagattcggcaagctcaactccgcatccccaccaccaccaccaccgttgaaccctgttgcctctcccggtgggagagtgtgaggagagggtatgagatcccctgggatactgcggccacacccatccctcccaacccaaggagagagggttaagggttgccataggaaccacaccagccagaggaggaacctctgggcgttacctcaccccctacctcccaccccctacctcccacccccccccttactccgcggagatttagaatcctggggtagtctgatagtgcctggaagacacgaggaaggggcccctcatccctgatgcgagcatcattctcttggcactggcaaccatagccatccagagagagagagagagagatgcctctaatgtattttattataagcgtgctgactgatattattttattaatatcatttaaatgagggactttcatttttaaaaaaatccacatctacaacagcccttgaaaccagagagggatagaaagctaagaggtcccagcctccccaccgtacagtaagctcggtcttcgaggttttctggttaattttaccgctgaatactggtgcgggtaagcagttttgagaccgatggatgagcttcttaagggccgaattgcccacgtcgactgctcactcagattgatgtaggctgggggtggaacgccctctcccgatcaaaccgcggcctgaggctgtggcatcgtggccgggcccgcctggcagaaatttcgcgagcatctgaaagataggttcaaatactacccatccctcgccacacgacgggctgctgggaacaggattcttaagctagaagaagccaggctcttgggctggatgcggaaaggggaaggctgggagggggggctgactatagcgggcccgttgggcaaaggtgtcacagcggaaaaatcagcgggagggggaggagacagaacgttttacccagtgcagaaacaaagctcagatcacagcgtcttcgaactcccattttgcactgtgttgaggggtcgagtgaggtactgtgctggccaaggcccgtgttgttaattaaagatcgtgggggttgctgctgagtttgagagactccattcctggctctgccctttaacaagccagtttcattcacctcagaagctagaagtttgtgctgaagccagagctgtggagcgctgagctaggctcatccgaaagggttcaagagccaagatttcacccaccaacctccgcgggtgtgtgctcccttttatgtccgggaggccctatttatcctctagtgttcaagaaattgtaatcccaagtctttttttccggctggttagcattgggtgtatgacagggtcagagacaaactaaccaggccattttcggctcagtccccgggctccacaagactgcccaaaattttcacggatcctacgccacccattcaactgtcaggtctcatagcagaaaaagaagtatgaagccatggtaggttaattcggtggttacaattctgagatgtcatctggggttcaatgatactgcttgtgtttgccacataagaattgcttgcccactcgccatttgcaaagcttctctaaacaaatggctgtccagataatttgtgtgcccattcctcccccagctgccaggccgggtgtaatgactgctgatctgttctggacagtttgcctcaccagcagatgtgtatgatatagtaccctagcttggctttgcggttcaccttcggatggtttgaatatttcttagcttggctgggtggtttgtttgttttttttaaagatgaatgacagcccacggtgtgcctctttgccctctgcacagaccctgtctgtctacacaattgtgtgttggtggaagggtgtggcttttaagtggcttctagtgtcaggctccgttccccagagaggaacaaaggaagcagagagagtgcccaggaattgcaagaagaaaacttggttggattgagttaaggtcaggcttgctcgttgagaacatgagcccttgtacagagcaagctctctggcccactcgtccttggagggctgggtcagcctggggatgcgatggggctggagtggaatttctgcagactgacttctcctcccctttaaacatcaacgtgtgcagttcatttgttggaaatgttaaggacttgtgttgcattccgtctcccctgagcatactgtatatggctcttgtgagttcctggctacgtgtatgaacatacattgctttttttttttcagttgaatatatgaacctttcccactatgccttttcttatatttatttagttaagttcttccttggtcccctcttgaaaatgaatcacttcaaaagaatgtttttagatatctaatataggggctagatatagagtgggtgtctggcccaccattgtttgacagatccgtgggtaaaatggagagatacaggtcaggagggcatccaagagaacatagagaggggcaaatattcaagctcacagcatccgggaagctcggggacaaatccagtctattttgtctatgtgtccagttggtgcccacaccgctgtctgtgatctgctggacacctttgctctgcctggggccccataatgtgcacaggtggctctcctgccccttgttgttgaaggggaattaagagcctagagggggctacctgtgggggtggccatgttccggctgcaggtgactaggatggagatggggagcctcactctctcatcagccactgtagtgacactcattggattcctctgcaagcaggagttaggggtacctggccaagtgagaaggagaaaagtccctggctcctaagcagggcctcataaggctcctgagacttgggtcttttcctggaaaaccaaggtcctcattggggttgctgaagtatctggaaagtggggtgtgttgggggcatcttgtgggtagaggcgggaaatgctgctaaacattataggacagctgccaaatgtcactcactggctctggcatagagaagccctgctgtagaaacagtgttttgctggcactgttatctcaattgtatatagtgcctgtcttctaggagtgctgcatgctgctggagatgatctcatatagttctcactgtgaccctctgacacatagggaagccgaagcccagagaggtttcataaccaactaaggatgcacagcaaagaacacacagctaagccccaggagatttaatggaactttgtgggctagcctgggaagctaatcgggctttccaaccacccagttcttttgtaagagtctgattggctcacaggctggaataagatacggaagtgtggatttgagtccttggtgtctgctttggacatctgggacaaaggatttccctccaccttggtactctgcttatcaaacagagagcaagtctttcccggaaaccttaacccgacctgactgagcgattctccactcatctgtttcgtgaagggccagccaggtttgactggcttaaacataactgttaattaagataggggcatcaagggcagggaaaatggctgtcccctcgcatgagggccttcctgagttcagatctctagcgcttacataaaagctgagtcctgctgtatgcatttattaaaccattgctgggcagggggtgaaaacaggtgagtccctgaaactcactatccagaaagcttagccaaattcacaacctctgggtctctgaaaagcagggggggggggaggcacagagggggaggggggagagtgagtgagagagggaggaagatatcagttggataattggattcagctgtggccagggtgaaatcactgaggtttggggggtgtcagattcctgtcttagattctgtccctcccatagcttgaacattggcacccatggctgaggcccagccctgaaactgtggatatatgtggagctgtgagcacaagaagctctcgttgcaagcatcccaagtctccgtgggcatcgtgggccctgcaggctatgcttgctgcctggccagactccaaagtacatttgcattatttctctgtgccacagtgccactgagaccgcttcagccatgggctcactgctgctgaccgtgagaggtgaggcagccgaggtgagcaggcccctcctagcccgccagtgccagcaatggaacacatactgtcacacttgcttcactactgtcaccttgccaaagcagttgctggcaaataattccttccccgcctccacttgctagacttctgcctgtagggctcccagggaacatggggggcagtgttgaggttacccccagctagctgtggcattctagattgttctctaggcatgtggatagaatgggcatctgcaaaatgaaacctcaagggacttccaggcgggtatcttaacctttcctcttcgctcatgtgccagcaggctcttcactgagcccaatcagcttctcaatagaaaatcgcaggaagtgtatctcttacctgagcaattcagagaagtgttgtaagcgcctggagagcaggtgcccagcacagagggcatttagaggtcatttggtttagaacccttggatgccagtgccagcttcccaacaatccaacctatggtggagcttagctacttagtaactggctttttttatttttaatatattcatatgtggcctggggtattatagggcctgtccagaagattgcctaggagtttaaatgaggaaatgtgatgcttgccatggcctggcatgtgactaaatggcattcttaaccatctgtatcctgggccactggtgttggaaaacatgcagcagataaatcgcaccaagaaagaaccaagcccagatcctctttcggaaccaggcattgttgtttgggcggaagtggatctccaagatcacacacacccatgtttaagtgtacacacagaactcttgtgtacagggacttatctgctgtcctgcttctgtctgcttccttcctgaatcctgctccatgccaggattagactgcaaactccccacaccagccttccctatacacccactggcgctctctccctctgcccccccctccctccctccctcatggaaagggtgacctggctcagtgtgttaggcccgaggtctcctggtttactcattgaagcaaagtgactactggtgactgagagcgtaatgacagtatcaaaaggcagtgcgggggggggggggggggtgagattgcaaacgaagtggctcccctcctacagaagtcagtagcccggtgagtggccttcatggctgacagtgtcaagagagacaaatgttaagtaatccatagtcctgagaaatgaagcctgggaattccaggcacccaataaaccgagtagagtgtgagccaatagaacaagccacagaagcctcctcaatgtggaatcaaggccaagggcgtaatagagcagttttggatcatttttaggaatcatttttccgcttcatttaattatttatttgtgtgtgcgtgtgtgcatgtgtgcatagatgagtatgtaagggtgccctcagaggtcaaaacagcatcaggtctgacctcctggagctggagttacaggtggttataagccatggatgtgaggaccaaacctgagtcttctacaagagcaatacatggtcttaaccacttaccatctctctatccccttgaacctttaaaatacagtgtgtgtgtgtgtgtgtgtgtgtgtgtgtgtgtgtgtgtgtgtgtgttgtgtgtttatttactgttgtattaattatctaatctcactcaggggtttctacctgtattagtcagggttctctagagtcacagaacttatggataatttctaaatagtaaaggaatttattgatgacttacagttggcagcccaattcccaacaatggttcagtcgcagctgtgaatggaagtccaaggatctagcagttactcagtctcacgcagcaagcaggcgaaggagcaagagcaagagctagactcccttcttccaatgtccttatattgtctccagcagaaggtggagcccagattaaaggtgtgttccaccacacctttaatcccagatgaaaggcgtagcccagattaaaggtgtgttccttaaactcggagattcaatcttctggaatccatagccactatggctcaagatctccataccaagatccagataaggatctccaagcctccagataagggtcactggtgagccttccaattccgaattgtagttcattccaaatattgtcaagttgacaaccaggaatagccactacaatccaccccttgtcaacttgacacaaataatatctcatgttcacatgaaacaataacaaggttgtaaatacgcctaacatgatataactatccctcgtacaatcgcaaacgcattagtaaatttacaatgggcattcatattactttataatcctcgtttctgcaactggttacgtggccttaattggtatttataactaccttcctctactacccattctgtatttccttcaccttcagccagcacctcagcaggtcttggctcttttcctggaggattgacccataccttcattcctgatgggtctgcgtcctttgtcatcctgcttggattaggctgttgtagtttcccattgactttaatcacaggacatggtagtactaagagacgccctaagggatctcctgcactccagacataatcttgcttaccaccattgtgaagaggtaatccaatttccccatggtaatctggatctatcacccctcctaacactgttattccttttttagcctgttggtttaagggcattagaagcccaaaatgaccagggggaagtctgagcttccagttcaatgaaatgtttgttgtagctcctggtaggagcactcccctctctggagccaaaacttctaagccagcagaacctagagttatggggacaggaagcaaaaattttcctagagggtcactaggagtgatagtaagtggaactattccgttttccaccccttgattcctggacccatgaatcctggctatgggtgaaactgtaccatatattgagcgctgattcaaagcatatactgccttctgaagaactctgccccagcctttcaagctgttaccacctaattggcgctgtaactgcgtcttcaaaaggccattccatctttctatcagcccagctgcttcaggatgatggggaatgtggtaagaccagtgaattccatgatcgtgagcccactgtcgtacttctctggctgtgaaatgagttccttggtcagaagcaatactgtgtggaataccatgacgatagatgaggcattctgtcagtccgtgaatggtggttttagcagaggcattacgtgcaggaaaggcaaatccataaccagaataagtatctactccagtaagaacaaaacgctgtcctttccacgaaggaagtggtccaatgtagtcaacctgccaccaggttgctggctggtcacctcgaggaatggtgccatatctggggctcagtgttggtttctgctgttggcagatctggcaatcagcagcagctgtagccaggtcagccttggtgagtggaagcccatgttgctgagcccaagcataacctccatctcgaccaccatggccactttgttcatgtgcccattgagcaatgacagggatggctggggagagaggctgattgtccacagaacgggtcatcttatccacttgattattgaactcctcctcagctgaagtcaccttttggtgagcatttacatgggacacaaatatcttcacatcctttgcccatttggagagatctatccacatacttcttccccagatgtctttctcaccaattttccaattgtgatctttccaagtccctgaccatccagccaatccattggctacagcccatgagtcagtgtataatcgtacatctggccatttcttcttgcaaacaaactgtaataccatgtgtactgcccgaagttctgcccactgtgaagatttcccttcacctgtgtctttcaaggttgtcccagaaaggggttgtaatgctgcagctgtccacttctgggtggtgcctgcataacgtgcagagccatcagtaaaccaggctctagtcttctcctcttcggtcagttgatcatagggaacaccccatgaggctataggtgcatgcttggcagcagatggcattgtaacaggagtagaaaccataggcatttgagcaacttcttcatgtaacttgcttgtgccttcaggacctgctctggcccgatcacgtatataccacttccatttgataatagactgctgctgtgcacgtcccactttatgacttgcagggtctgatagtacccagctcatgatgggtagttcaggtcgcatagtgacttggtgtcctattgtcagacgttcagtttccactaaggcccaatagcaggccaagagctgtttttcaaagggagaatagttgtctgcagatgatggtagagctttgctccaaaatcccaaaggccttttctgtgattcacctacaggggcctgccagaggctccaaacagcatctctatcagccacagacacctcaagtaccatcggatctgctgggtcatatggtccaagtggtagagcagccagccagtaaaatcatttttcgattttttccccatcttgtagaaagctttgtgcactgaatcacctaattcattaagaaaatcaagggcattagcttctttaagttcggaatatagtttcaaccatgggtcttcaaaattctctgagctcccaggagggagagaatctggagaggtttcaatatttgaaagtgctggtggatcaacaagccaattccagtattttaaaagattcatccttgtacttctgttactctagaaccactcctggtaccaacttctgtattagtcagggttctctagagtcacagaacttatggataatttctaaatagtaaaggaatttattgatgacttacagttggcagcccaattcccaacaatggttcagtcgcagctgtgaatggaagtccaaggatctagcagttactcagtctcacgcagcaagcaggcgaaggagcaagagcaagagctagactcccttcttccaatgtccttatattgtctccagcagaaggtggagcccagattaaaggtgtgttccaccacacctttaatcccagatgaaaggcgtagcccagattaaaggtgtgttccttaaactcggagattcaatcttctggaatccatagccactatggctcaagatctccataccaagatccagataaggatctccaagcctccagataagggtcactggtgagccttccaattccgaattgtagttcattccaaatattgtcaagttgacaaccaggaatagccactacactaccctaccttagatcattcagttcccaggtaaacaacccaaagcttttatatttataataagccttaatcagtactaaagctgggcagatatcttctctcgactattcctatctacttcgctagccataaccctgaattctaacttgccatgttccgtctgagctgctgctcttaatttcaattggccagccctcatggccatgttttcatgactcacctaccctatggcatctcctcttctctccaccttctttcctcaaggtccctgcctcagaccccagcctgggaaccaaaactccacctatctctctcttctgccgagcagtaggctgtagggataatttgtggggcggggctacatagcatcacctgggtctgctcgaggtcctcctccttcctgggggcaaccagggccagtatttagcattacaatagcaacagaccaaacctcaacactttgtgtgtgtgtgtgtgtgtccacatgtgggctcacagaacaatttgtgagggtcagttctctccttccaacgtctgagtcccggacacaaactcgggtcatctagtttaggaccaagcacttgtttggccatcccactggacctatttataccttttgggggagaaaagtcttatttgtgtgtgcatgtatgtgcgtgcttctatagtgtatgtgtatgcatggtatgtgcatgtgtacgtgagatggaagccaggagacaaaacatcagattctccatacattgccatccaccttatttgtttatttatttattaaaatttttactttattattttattttatatgtatgtgtgtttggtttgcctatgtgtgtgcaccatgtatatgcccggtgccccccacagaggccagaagagggtgctggatcctctgggactggacttatagatgtttgtgaactgccatgttgctactggaaatcaaacccaggtcttcagaagagcagccagtgctcttaaccactgagccatctctccagccccctattagtgtttgaggcaacatctcttgttgaatccagagctaagtaattgactagaatggctggttcatgacatctgggatcccctgtccttgcttgagttcaccatgctggtgttacagatgagcatcattggtgttgcgaatctaaaataagagtttctagggcttggtgggaggtggcttgggaggtgaagtgtttgccacataaacataaagacctgaattcaaatccccagcgtacacatacaagccagatgaggcagtgtggttcctcgtgctttcaagactctccccagcctctagcttgtacttatagaagtaatgcaatcgttatagcaaagatataatagccacgtgatggttccagttacctgattagtatagcaaataggatagacagagaaggcttcagaacaagcccagatgaccctgagcaggagcttggttgtggcaggaggttggcctggctgcagacaggctctgtggatcccagccttaagaaggtctttcatttctcctacaaaaggccagaaaagcacctaccagggagacatttgtgttaagttaaaggcaggtatactcctctctgcccactcttgcctgtctttaaggcccccagaatacacatcagacacaactttaaactcctcttaggaaaaagttttattcaaggtccaacaatagaaagaaagtttgaaatccacaagaaatgaatccgtagtcactgataatggtggcacttcctctgtggcccttccactccctgccagcgaggaggagagggacatctgtgatctctggtccctggtcttccttctccttccagcctgtggctcagtgttgttttcccagaaagagtgggtgatgctgtcctggtaccctcgccctagcggtgacacagaagatccaggcatgtactttgatcttgatgtcttcctccctgggacacaaaggatgtcagaaaacctctgtgtccataactgcctgtcaattatgtcaattagtctgcagccagcataaagtgataagcagggagcgctgggaagactcacagagggtgccttctccaggacagctcttccaccagaaggcttcaaactatgtgtaaacaaaattcaagggggcttaccaagcaaaaacaaatgaagcaaaacaaactaacaaaaattcttaaaattgttctctgtcaaatctagccctgccattcatttacactgcaattcaggaggtgtgtgtgtctgtgtctgtgtctgtgtgtgtgtctgtgtgtctgtatgtatgtctctgtgtgtctgtgtgtatgtctgtgtgtatgtatgtctctttgtgtatgtctgtatgtctgtgtatgcatgtgtgtgtatctgtctgtgtgtgtgtctgtgtgtgtctgtctctatgtctgtgtctctatgtgtgtgtatgtctgtgtgtgtgtgcgtgtatgtgtctgtgttgtatggcatatgctttcacatatgtgagtgtgtggaggccactggctgaggtgtggtgtcacccactatggccccacaccttatttttaatttttattttattattttatatgtagaagtgtttgcctgcatgtatgtttgtatgccatgtgtgtccctggtgcctgtagatgtcaaaagagacatcagattacctgagagttacagatggttttgagctgccctgtgggctctgggagttgaaccaggtcctatgaagagcaggcagagctctttacctaccaagtttcctcaccagcccctccacaccttactgagctcaacattttcattagaggagtgactagccaacaggatccatctgtctgtctgtctgtctgtctgtctgtctgtctgtctctctctctctctctcacacacacacacacacacacacacacacacacacacacacacacaccgacccatccagtgtttcagacatatgctaccacagctggtttttatgtgggcacagggattgaactttgctcttcatgtatgagaggcaggcactttagccactgagccacccctcccccactgagcctgaggaacattttttttttttttggtttttcgagacagggtttctctgtgtagccctgactgtcctggaactcactctgtagaccaggctggcctcgaactcagaaatccacctgcctctgcctcccgattgtgagtgctgggattaaaggcgtacgccaccacacctggcctgaggaacacttatatgtcccccaaattacttgtcttaccttatccttccccggtggccttaccccccaaaagaggtttgccgatgagctaataatctgagaccagtcctaagagaaggttggacttaaagactgactttggttacacagccataaatgtgaatattgactattgtatgtctggggtttttatgttgagcttgctgaaaacctggtcactttttaactttgttattggcagtgcatcagaatttaagtaaatttatgttaattgtcagaggaaaagttggcactgtgagtaagacagagtaaaccacaggatgcctctagggcacaatgaaaacccacagcgtggacaaataggaacaggtctctttcaaagctgcccattgcctggaagactgagacagcatttggaggtctcaccagcctacttcctgcctagtctttttccagaagaacacaggaaatactacatggcttgaatctgctgtgactactgtggggtcaggctctgccccgcccctatgcagccagctcaggaggatagactttgaaaaagaaaataaaaatccctaaatccgcttaaacgcttaaacgtggaagatcttcacactccccaggaccttctttaagctggttgaatattctcctccaataaacctcttgggaatgatatgcctcccaggtggctcagtgctggggtgagcccctcctcccggctccaaagtccagcccttcctcctcctccctcctccttcctccctcctccctgtgcctgagaaaaggctagtcatggatgcactgttccctgcagagtgctaataagggctgagtgcattaaaatatatgccttgctttttttttttggtatcataaaatattattaacaatcacaaggcagcccttaatggctgttggagaatttcagaaaaacatttctttggataagcaggatttgtgccataagttgacagtcgggaccaaggcttagaataagatttaagacttgatccatcatttgttgctgacaaaactgaactacagaagccagtgagctgctcacggtgtggtcttggtcatcacccactgctgcatcctgaatttaactgccagtttcccgttggccaataagagatttttgacaaagatggagaattcctagacctctgtccttcgtctgcaaaatgtgaatgatacagtctccccttggaatggagtggatggtaaggcctcagccttgatgtctactgagtagtcacagaactggaatgaaacagtccatgagaaatccttggggtagctcagaagtcagtgtcccactggaactgctacagctccctgatcagaggtcccaagtgggctgagtgtagcctgaagtggcacagctgctccctgccaccttgctcctgtttccagccagccggaggcagctggtcctctgtagtgaacactaggtcagcacgggtccatgtccagaaggataaacaggcggccacagcccagtctgtccccagtgtagagaggaggagaccccaagtcagggtgcaggacagtgggaggggtgacctcaagtgatttcgagacctctccggaaaggagctgggacttcctcttggtctccagttgtgacccttgagtggaatttcctgggagtgtgcccatttttttatggaactttcagtgtcttcctaattggtacagacttgacacagaacctcacattctaaattttcccagggtggattctccctgcctgtccatggtagcatatgacagaaatccaccaaggtgagagcaaattcgagccagaccttgactttgatggcatgatacttttgtggctcattaactgaatggctttaattgagccgcagctgggctggagttcattgacttagccctgtgaacggaaggtttctttggcctcgtcctccaacccccagcactggggattgaactcagggcttcatgagttctatcacagagctacatctgctgcctgaatggaaggttcctgaagaatgaggctggtgggaaatccagtgtgcaaccccatcctgtcaggggcattggctgcacctcagtgagctccagtgtctactgaccccacgtcactgctccttagaccctcaccatctgatcttcccgcttgggctgttgaaaccttccatcttcccatcccccgagtggtgaaatgcagtcttcagctctccctccatatcctggagtggacctgtcggtttcccgcagacattgcccacccagctttgtttctccagctctgcttttactgactccaaaatgagttcttacagggctggagagatggctcagtggttaagagcactgattgctcttccagaggccctgagttcaaatcccagcaaccacatggtggctcacaaccatctgtaatgggatctgataccctcttctaatgtatctgaagacagctacaatgtactcatatacataaaataaaatcttaaaaaaaaaaagagttcttaccctggcatcgttatagaaataattataattcctagagtgaattctaaagttatactcgatgcagtttgtatcccattgccaaacacttcccagagcaaaattctgtctgtcatgttcatgtccacccacaaatcccttgtctgtcacctgctaagccgatcctgccctcacgccccgcccagaccctggatttcttctcactgccagccagctgccatgtcttcttcatagatggcaccttcttaattccctcatcttccatggcattgtgcaggtggtaccctcctcacctgtacagactccgcagtctctgtcccctctgagttgtccccacctgtgtgcactagatccaggagccccacacacttcctctcccatgcattcccactggagtagcttatgtctagctgtcatcctctttcttgtcttgctttgtgtgcatttcctgcctcctccaggttatctgtcctctcagagcagacactgtgcatcaccaggcactcctggacctctggggcataccacagtcacccaacccgcactcagagccccgaacgttctggttttgtttgggtttttgttttgttgttttgttttaagacagggtctcactctggagcctaggctgacctgaatctcactggatagtctaaggaactttaaacttgtggcaaacctcctgcctcagcctcctgggtgctgggattacaggatgtgcctgctagcacactctgggtgaatatggctatgcaggctgatgctgggtgagcctgacctgtttaaagaagacgagcctttccctgctgatccacagtcccactgctctgagcacctaaaaataaaaatcacattttcagtgagatacttgattgcagtctgttctactttgaaccagccaatcaaccagtctcccccgtgatccagatccgttatctgtatgaagacgcacagaacacagcaactaccggttgcatttattttactaaaaactgtgccttggtccccgtttccccacgagactgggtcattttgcaaaggagcaaagaaatttgggggcttaagaaagagccaaatggtgtcttaaacccaggtctactccatgtcaggcccttgggtctcctccccaaggctccatgctctctgccagcgccgcgaggagccagaggacctgtctgtaagaatctgggaagcatgcccacgtgaacgggtcacttgtaacgtgagcccggagaaattattttaacctgaaagcaagcctctaaaccaggctgctgcttcatgtggcccatgacggcaaaaccaaatttagcatcacagacaggtccaccctgaacaaactccactctcttaaaaaagtcttccagctcacagaaagcatgggcaaacattcacacccaccacagtgccaggaggttcccagtgatcagaggctgggggatgagcgcgtaaatgaactcagggaagagacagacagtgtttccaggaggagagtcagttaaaggggacaggttccctggatgcgacaaaggcatctttgctgctgggggaaaaggtgggttgatgggacagagggagaccccgtggtccttctcttttaaaggtcctgagttcgattcccagcaaccacatggtgatacataaccaactgtaatggggtctggtgcactcttctggcgcatacagatagagcactcatatatatattaaataaataaaaataaaatttaaaaaatgtaccataaccaaaaggcacagttcctggagaccacagtcccacttagtgggtcctgtggcgctgcagaatgggtgtctgtgccagcccttatgtgtgcggcagcaggtacaagggtgtttgttacaatggtccactagataacccacagaaggctacgcacattcgtgtttggggacctcattgccgaaggtcttcacggtgactccaacaatgaggtcccacttgctagtaggacaaaccctgcccccatagggggccccaaactttcaaagaacacttgagaaaaacctttctggatttactaaaatttggtgtgatttgggatgaatttagagaagacgagtcattttaagttgtgggtgtttgagatcagtgactgacatgggtcacctaaagtaggcccaggagtagggcagtagtggtgcacacctttaattccagcactcaggaggcagaggcaagcagatttctctgagttcagagccagccagagcaacagagtgagaacctgtctcaaaaacagaagggaggcttcggaggagtgggcggagcctgcgggactcaacagaagacaccaaaagaagaaagagccaggtctttctctgtgtagccctggctgtcctggaactcactctgtagaccaggctggccttgaactcagaaattcacctgcctctgcctttcaagtgctggaattaaaggcgtgtgccaccaccgcccggcctaagagccacatcttttttttttttttttttttttactgtatataattatttatttttaatttaagtcaaataaattatttgagggattttgattcctttgctgtccatccaggtcacctgaaagagaagacaatgagagatgtgttaatgagttaagtgattcagggaaatgtatgttatcctctcagaaaaacacagcactatcaaatcagtgcaggattttctctgtgaagaacatcaatctttggtcacagcgttaagagctgtgggttttatagcccaaaaagccctctttgaaatgacctaagagccacgtcttacgtagcacagttttaggttttactttaaagaagaaagcaatctgtgtctttacttatggtacaaactctcagatctctgaccaatccagccggccaggcagccaggcaggaaccatttcataacataagttgtgcttgggtgtgggtgtaatttgtgggtgcagtgttcccatagtgttgcagtggaggataggtgagaaaaatcccactgtccaggcagccccgaggagctgaagaggagcaggcggagactgcgaggctcatcaggctgccctgggaagcaagtgactgctggggctggggtggtccaaggaagctgaggtcaaggcccacactcacccatatacatctccaaagatccagaagacatggaaggggaaagagccacgtcatcaggttaacgaagccagttaagtaactcgtcttcagacgcaagttatttttcccctgaaactaaagtctaaaaataggtaaattaatgagtgtctgtctatttcaaaccaagttgatttctcccctttccctctgctatacccattgtgtgtaattgcagtgatagcatctcataccacggttttaaaccaccctggggaacaggctccttagctaacccagtatgttgaagggactttcacccatgtgtccccagcctctggcattctgcagggcacagaacaagagctaggcaccatggaatggatgaataaatagataacggatccatgaatgaacgtagtatgagttaaagcggcacacaggggaaggacaatgtactttggtctgtgtgttcgtctctctaccttctggagttctgcatttggatttggcatgctcacagtagaaacatgtgcacagtgtgtgtgtgtgtgtgtgtgtgtgtgtgtgtgtgtgttacaactgcctttggggatttcttgtttgtttttcaagacagggtctctctgtgtagctgtggctgtcctggaatttgctctgtacaccgagcttggctatagttgtctgtttaaaagttcagtcaggtgcctttaagttcccctgaatcctggaatggcatcatgagccataagtcccacatcagtggctgtgaacagggtcgggtctgtggcctgggttactggcattccagctgactcctagagtttcagttctgcatagaggtgttagattgtcagatttgggttggagagatggctcagcagttaagagcactggatgagcttctaaagggcctcggtttcattcccagcactcacacaatggcttaccactgtctgtgacttagtcccagaggatacagtgccttctgctcacctccaaaggcatggggcatacacatggtacacacaggcacatgtaagcaagacacccatatcttaaataaataaataaataaataaataaataaataaataatttttaagcagctggaattgccaaaatagaacacagggtgtccaagagctggagagatggctcattggttaaatgatccagaggatccaagttcaatgcccagctcccatatcctgtaacttcaactccaggggaattctcttctggcctctatggccctgcactcctgtgcacataccctactccctcacatacatacccataactaaaaatataatctatgaaaaaacacacagggtgtccagttgaatttggataccaaaataataataataataataataataataataaagaggctacaaaatgtcccctttagatttccagaggagccttcaagggaagtggatcaaaatgcttctatcatttcctgtcacaccacacacacactgaaaagccctggactggaggccactgttaaagactgacagccattttctcatgtgaagaggccttccaagccaaggtgagcacaggccagctcagtaaaggtttctttctgtcattgtctttcacacccgatctgttccccacactcaaaggatccactgttctggacacataagggctctgggatcatccagaataggccatggtgctaagaccccaggattactctccagataccaagggcttatagaagagaaaatgtacctggtggaagaaagagcaggaaaggagaatgaacacgaaaagctatttggtctcacctataataggtggctggtccacaaggtgagatgtcctcactgttcaccagacagcagaaatccagaatggtgtgggcggtgactgcagcttattaaccaggctgcagcctttgacagagcctcagagtcggagaggaactgcctaagagagtggtccttaaagtagtctttattctccaccccaccccaccccgtgtatgacctgttaaaaagataaatatgtacaccaatgcaccagctcacctcagaggatatatctgttcgaagcatctagctggattgattgcttggtgggtcacatctgtaattatagacttgaggtgctaagtaaggattataaattcagaatcaacccgggaaacacaaaaagaccctgtcccagataacactcttaactccccacaaaagctcaaggccttctacaaagactgttcttttaaacacacacccctccccgccagtggcatgcctgggaagaggttctgatctgccctaaccatcttatgtcacacagaaacagggaagcggttgcaccaggcccctgccagctatcctcaacacttcactgccttacacagaaccttccagttaagccacaggatgagactagccgaggagcagccaggcatgcttgtacacacctttaatcccagccacttgggaggcagaggcaggcggatctctgtgaatccgaggccagcctggtctatagagtgaattccaagacagctagggctaccaagtagagagagtctgtctcaaaacaacaacaaaatagcttagaagcatggcatacatgggtatgtacatgtgtctgtgtaaaacatgtttataacataagacatagttgaacccgggaaccaaatgttcttgtagacccacagatggacgggataggtgagcaaggagaaatgggttagtgtgtttaagtccctaccttaacatagatgggaacgaacgatctacactgcccagtggggaactgcatgtcatgtgacaacatagatggaactatctgatttataatcccaaatcctttttatgtcaaaaatagccccactgctattttttcctcttggtgctttcacatagatgacttcattgcgtgcccctccaggccgttccagagccagtggcacttcctgtttggaaggggaggaaatgggtgctgcctgtctccatctccctaccttcctgggtgcagatgccacaccctgcccctgcacagtcctcccccaggcagctcatcatggtgagcgggactccttcagaatccctgcctgctcacatcacccctgcttcctctgaaggactcagagaacatactggagaccctgcggagttaaaggagccgaggcgatgtggtacctcagtgttctccaagtctttagcaagtacacgcggttaccagccttggcaggctagctgagcacctggcagcctcagccgggagaaccttccagatgggctgtaggcttccagcacaggtgccgagaatgatgctcttcccaggctcagacccttgggctggtcacccatgtggtccaggtcccaggtcttgggcccatttttggaatttctatttggagatgtgggggtggccactgagcatgggacagatggtgcattcctggttcagctcattgcctcagcacaatgagatcgtgccagcatgtttgcagatgctttctggttttctccttaaggaagtcgagctccaaagtggcttgatagctttcccatgggcccttagctcatagcagcctcactgggcagtgcaggggctgcttggcctttttcctgtattgcttcctgagagctctcctcccatccttgctctggatgctctggtcccccaacccagcacccatttaccagaggcctagcattgccctttgacccttcccagtcctccaaggaatccagactgtagccttccagtaggaatagggtggctgtgtggatgccttcctgtgcattggaatcccacaaacacaccacacaaacccatcatctgcaggctgcagaggacacgtatggagtccacaagacacggaggcacatctgcagagccctgctgtgtggcccaggtgagagaagcaggagatggaagagttctagacactgactggtgtgcagctgaggcctgcctggccctcagcgctgtccagcaccacctaacaacatgcatggatgtaaacttcacacaggattgccatatagcgggacagagacacagctcaacatgaatccaaggaaatctgaatgaggacatttcttactctgtttccctccataggcttttaggtgaaacttagctggtaatatccctttatttgtcctaat

(19) United States
(12) Patent Application Publication
(10) Pub. No.: US 2021/0329894 A1
(43) Pub. Date: Oct. 28, 2021

(54) METHOD FOR PREPARING ALZHEIMER'S DISEASE (AD) ANIMAL MODEL

(71) Applicant: Shanxi Medical University, Taiyuan (CN)

(72) Inventors: Qiao NIU, Taiyuan (CN); Xiujun QIN, Taiyuan (CN); Liang LI, Taiyuan (CN)

(73) Assignee: Shanxi Medical University, Taiyuan (CN)

(21) Appl. No.: 17/125,368

(22) Filed: Dec. 17, 2020

(30) Foreign Application Priority Data
Apr. 28, 2020 (CN) 202010351931.1

(51) Int. Cl. A01K 67/027

(52) U.S. Cl. CPC A01K 67/0275; A01K 2227/105; A01K 2217/30; A01K 2217/056

(57) ABSTRACT

The present disclosure relates to a method for preparing an Alzheimer's disease (AD) animal model, including: selecting healthy male human ApoE4 transgenic mice, and intraperitoneally injecting a 4 mmol/L Al(mal)₃ solution at a volume of 10 ml/kg, with aluminum exposure for 60 days, and interval time of 2 days for every 5 days, to obtain an AD animal model co-induced by genetic and environmental factors which are interacted without being simply superimposed. The AD animal model established according to the method of the examples in the present disclosure can express the characteristic pathological changes of AD and has the characteristics of learning and memory impairment. The AD animal model provided by the present disclosure can be used for drug screening and AD mechanism research, and has the advantages of stable properties, prominent reproducibility, easy operation, and low cost.

### CROSS REFERENCE TO RELATED APPLICATION

This application claims the priority of Chinese Patent Application No. 202010351931.1, filed with the China National Intellectual Property Administration on Apr. 28, 2020, which is incorporated herein by reference in its entirety.

### SEQUENCE LISTING

The sequence listing titled “Sequence Listing” electronically filed Dec. 17, 2020 having a file size of 6198 bytes, and created Dec. 17, 2020 is incorporated herein by reference in its entirety.

### TECHNICAL FIELD

The present disclosure relates to the field of medical biology, and specifically relates to a method for preparing an Alzheimer's disease (AD) animal model.

### BACKGROUND

Alzheimer's disease (AD) is the most common and leading cause of dementia, which is characterized by progressive degenerative changes of the nervous system and clinically manifested as brain atrophy combined with progressive memory loss and cognitive decline. As a degenerative disease of the nervous system that has plagued humans for more than 100 years, AD is clinically incurable for unclear cause, with the main pathological features of amyloid plaques consisting of Aβ aggregates and neurofibrillary tangles (NFTs) consisting of highly phosphorylated tau proteins. With the acceleration of population aging, it is estimated that by 2050, there will be 131 million people with dementia in the world, and most of them are AD patients. AD has become an important disease that plagues human public health and social health systems.

The pathogenesis responsible for AD that is a multi-etiological complex disease has not yet been elucidated. Most AD cases are sporadic in nature and this neurodegenerative disorder occurs later in life, mainly after 65 years of age, which is called late-onset AD (LOAD); and early-onset AD is usually inherited in an autosomal dominant manner, which accounts for less than 1% of AD cases. Typical LOAD is generally considered to be caused by the interaction of genetic and environmental factors, and about 70% of the risk of AD is caused by genetic factors. Among genetic factors, ApoE4 is considered to be the strongest risk genetic factor for LOAD. Studies have found that as the number of ApoE4 alleles increases, the risk of developing AD increases and the age of onset decrease. One ApoE4 allele will increase the risk by 2 to 4 times, and two ApoE4 alleles will increase the risk by 8 to 12 times.

Among numerous environmental factors, aluminum (Al) is an important environmental influential factor that has been widely studied to induce pathological changes in AD from multiple levels. Therefore, by combining two key genetic and environmental risk factors related to the onset of AD, it is expected to prepare an AD animal model that undergoes an AD onset process close to the true onset process of human AD and has the typical characteristics of AD.

### SUMMARY

An objective of the present disclosure is to provide a method for preparing an AD animal model through the combined action of genetic and environmental factors.

In order to achieve the technical objective, the present disclosure adopts the following technical solution: selecting healthy male human ApoE4 transgenic mice, and intraperitoneally injecting a 4 mmol/L Al(mal)₃ solution to obtain an AD animal model.

Further, the human ApoE4 transgenic mice may express human apolipoprotein E4 subtype but not endogenous mouse ApoE under the control of a human glial fibrillary acidic protein (GFAP) promoter, with insertion of human ApoE4 gene into mouse chromosome 15.

Further, the human ApoE4 transgenic mice may be healthy male mice aged 4 to 5 weeks. Further, an 8 mmol/L aluminum chloride solution and a 24 mmol/L maltol solution are prepared with sterilized triple distilled water, then the two solutions are mixed in a volume ratio of 1:1 to obtain the Al(mal)₃ solution with a final concentration of 4 mmol/L, and pH is adjusted to 7.4 with 10% NaOH.

Further, the 4 mmol/L Al(mal)₃ solution may be intraperitoneally injected at a volume of 10 ml/kg, with aluminum exposure for 60 days, and interval time of 2 days for every 5 days.

Further, the steps may also include evaluation of the AD animal model, including a Morris water maze experiment and a new object recognition task.

Further, the evaluation of the AD animal model may also include determination of aluminum content, Aβ₄₂ content, and expression levels of Tau proteins and phosphorylated Tau proteins in the brain.

The present disclosure has the beneficial effects as follows: An AD animal model can be established by combining ApoE4, the strongest genetic risk factor of LOAD, and aluminum, an important environmental factor affecting AD, where, the genetic factors and environmental factors are interacted but not being simply superimposed. This model, which undergoes an AD pathogenetic process close to the pathogenetic process of human AD, can express the characteristic pathological changes of AD, has the characteristics of learning and memory impairment, and has the advantages of short experimental period, excellent reproducibility, easy operation, low cost, and convenient use and promotion. The AD animal model provided by the present disclosure can be used for drug screening and AD mechanism research.

### BRIEF DESCRIPTION OF DRAWINGS

The above and/or additional aspects and advantages of the present disclosure will become obvious and easy to understand from the description of the examples in conjunction with the accompanying drawings, where:

FIG. 1 is a schematic diagram of a Morris water maze according to an example of the present disclosure;

FIG. 2 shows comparison of escape latency (i.e., latency time in the figure) of mice among groups of a place navigation test according to an example of the present disclosure;

FIG. 3 shows the number of crossing platform according to an example of the present disclosure;

FIG. 4 is a schematic diagram of new object recognition according to an example of the present disclosure;

FIG. 5 shows exploring object time according to an example of the present disclosure;

FIG. 6 shows comparison of discrimination index among different animal types at different intervention measures according to an example of the present disclosure;

FIG. 7 shows determination results of aluminum content in the brain according to an example of the present disclosure;

FIG. 8 shows determination results of Aβ₄₂ protein content according to an example of the present disclosure;

FIG. 9 is a Western blot diagram according to an example of the present disclosure;

FIG. 10 shows a relative intensity of Tau-5 protein according to an example of the present disclosure; and

FIG. 11 shows a relative intensity of pThr181 protein according to an example of the present disclosure.

### DETAILED DESCRIPTION

Example 1 Establishment of aluminum-exposed ApoE4 transgenic mouse model 16 healthy male ApoE4 transgenic mice (KI), 16 ApoE knockout mice (KO) and 16 wild-type mice (C57BL/6J, WT) aged 4 to 5 weeks were selected, with similar activities and weights of 18 g to 20 g. They were randomly divided into control group (NS) and aluminum-exposed group (Al), with 8 animals in each group.

An 8 mmol/L aluminum chloride solution and a 24 mmol/L maltol solution were prepared with sterilized triple distilled water, then the two solutions were mixed in a volume ratio of 1:1 to obtain a Al(mal)₃ solution with a final concentration of 4 mmol/L, and pH was adjusted to 7.4 with 10% NaOH. The animals in the aluminum-exposed group were intraperitoneally injected with the Al(mal)₃ at a volume of 10 ml/kg, with aluminum exposure for 60 days, and interval time of 2 days for every 5 days; and the control group was given the same volume of normal saline (NS).

During the whole experiment period, the animals were bred in a barrier environment and subjected to a 12 h light:12 h dark cycle of illumination, where, the room temperature was controlled at 20° C. to 26° C. and the humidity was 40% to 70%; the animals were freely fed with SPF grade maintenance diet for rats and mice and autoclaved tap water; and the experimental table tops and the cages of the mice were wiped with a disinfectant every week, where, several types of disinfectant were used alternately.

### Example 2

This example was different from Example 1 in that a Morris water maze experiment and a place navigation test were added. In a 5-day consecutive trial, each mouse would undergo a swimming experiment four times every day, where, a mouse was placed gently into the water from four different starting positions of SW, W, N and NE every time, at which time, a computer software was clicked to start a timer to record the time that a mouse took to find the platform from entering the water, namely, the escape latency. If the mouse did not reach the platform in a set time (60 s), a handler would guide it to the platform to stay for 10 s, and the escape latency was recorded as 60 s. An average value of the escape latencies in the four times per day was used as a result of place navigation test for the mouse on that day. Space exploration experiment: the next day after the place navigation test, the platform in the NE quadrant was removed and the mouse was subjected to a space exploration experiment to test the memory retention ability of the mouse for the escape platform; the mouse was placed gently into the water from the point (NW) farthest from the original platform, at which point, a software was clicked to track and record the swimming track of the mouse in 60 s, and to record the number of times the mouse crossing the position of the original platform. A schematic diagram of the Morris water maze was shown in FIG. 1.

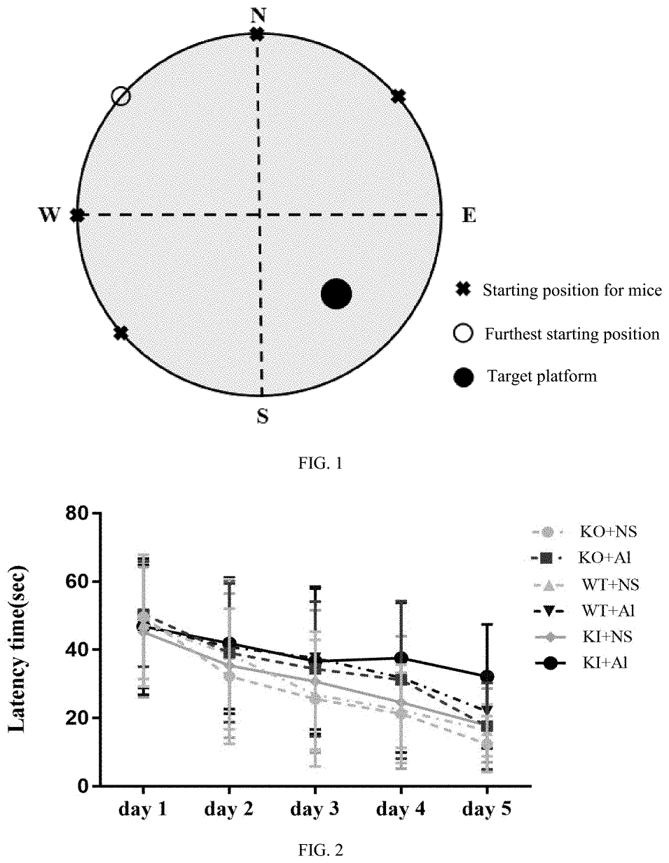

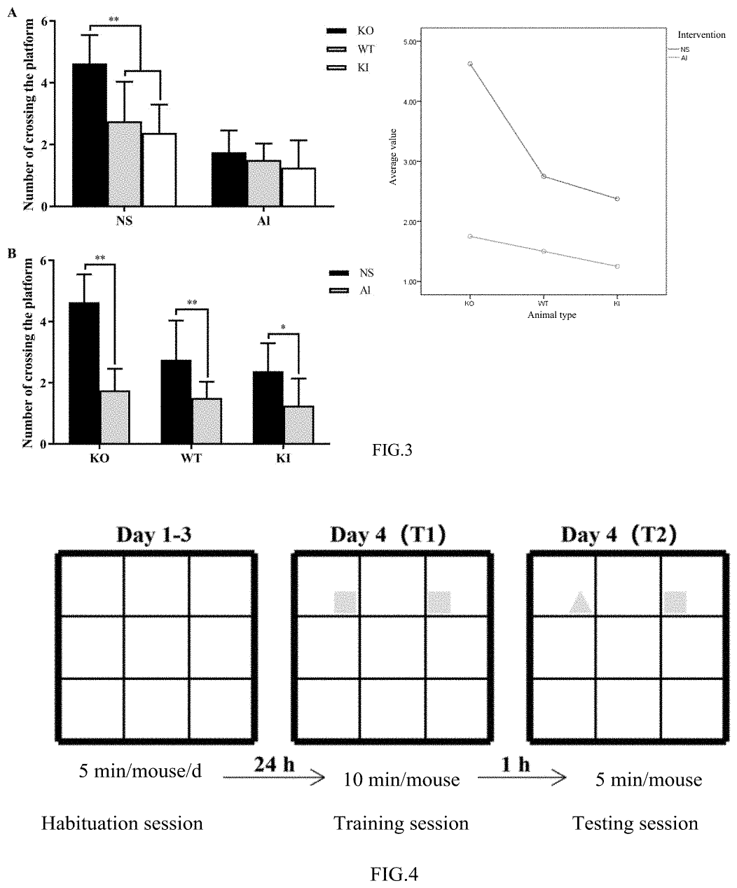

The results showed that the escape latency for each group decreased with the increase of training days. On days 4 and 5 of training, the time to find the platform for mice in KI+Al group (i.e., AD model group) was significantly longer than that for mice in the control group, as shown in FIG. 2. The results of the space exploration experiments showed that there was an interaction among animal types and intervention measures on the number of mice crossing the target platform, and the mice in KI+Al group had the least number of crossing the platform, as shown in FIG. 3.

### Example 3

In this example, a new object recognition test was conducted on the basis of the above examples. Training session (T1): 24 h after a long-term habituation session, two identical cubes were fixed in a space 5 cm from the wall of a box in a symmetrical manner (as shown in FIG. 4). The time periods for a mouse to explore the two areas were both recorded by a recording instrument, and the recording time was set to 10 min, after which the mouse was taken out and put back in the original cage. The box was cleaned with 70% alcohol, and after taste removal (2 min to 5 min), another mouse was taken out to undergo the above procedures. Testing session (T2): 1 h after the training session ended, one of the cubes was replaced with a cone, the mouse was placed in exactly the same way as the training session, and the time periods for the mouse to explore two different objects were recorded as a new object recognition time (New, N) and a familiar object time (Familiar, F). The learning and memory ability of mice was evaluated by a discrimination index (DI)=(N−F)/(N+F)×100%.

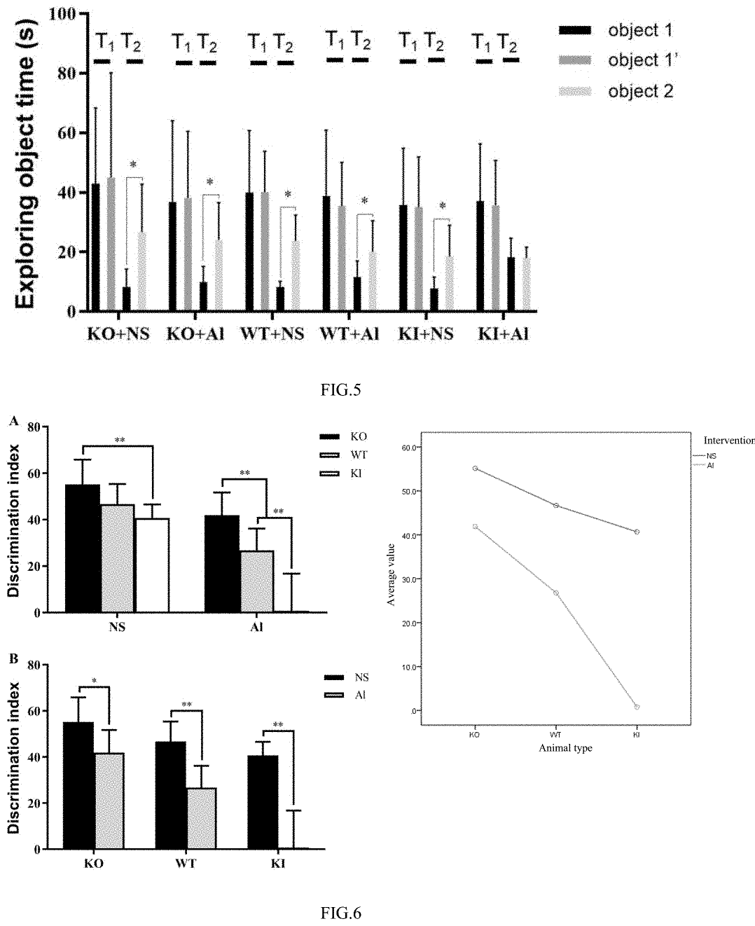

As can be seen from FIG. 5, in the training session T1, the time periods for mice in each group to explore two identical objects (object 1 and object 1′) were substantially the same, with no statistically significant difference; and in the testing session T2, except KI+Al group, the time period for mice in other groups to explore the new object (object 2) is longer than that to explore the familiar object (object 1), with statistically significant difference. Multivariate analysis of variance (MANOVA) on discrimination index revealed that there was an interaction between animal types and intervention measures on DI in mice, with the lowest DI for mice in KI+Al group (see FIG. 6).

### Example 4

This example was different from the above Example in that the determination of aluminum content in the brain was added. 60 mg of mouse brain tissue was weighed and put into a microwave digestion tube, 3 ml of nitric acid (guaranteed reagent) was added, and the tube was put in a microwave digestion system for microwave digestion according to a program; after digestion, the solution was filtered with a 0.22 μm microporous filter, and a resulting filtrate was collected in a 5 ml deionized centrifuge tube and then diluted to 3 ml with nitric acid (guaranteed reagent); and the sample was diluted 25 times with Wahaha water, and aluminum content was detected with ICP-MS.

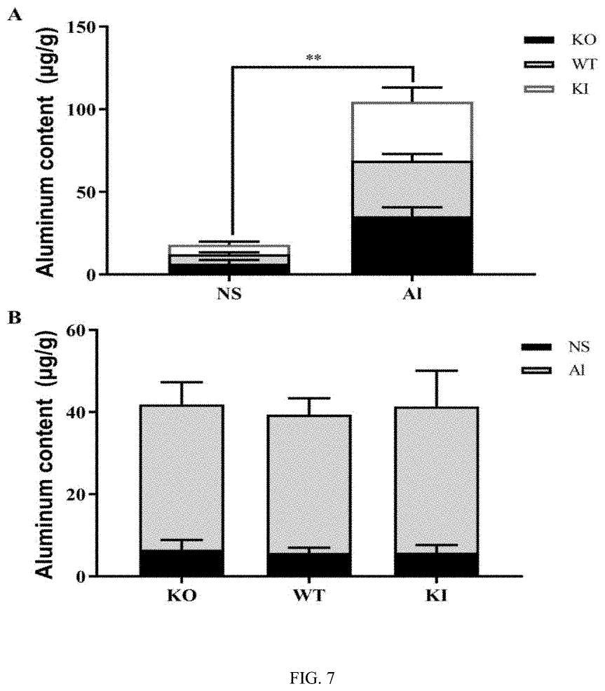

The brain aluminum of mice in each aluminum-exposed group was significantly higher than that in the control group, indicating that after aluminum exposure for 60 days, the aluminum content in the mouse brain was significantly increased. The results were shown in FIG. 7.

### Example 5

This example was different from the above examples in that the determination of Aβ₄₂ content was added. The hippocampus tissue of a mouse was stripped, and crushed with an ultrasonic processor; the hippocampal tissue protein was extracted with a tissue protein extraction reagent; and the Aβ₄₂ content in the hippocampus was detected with an Aβ₄₂ protein Elisa kit.

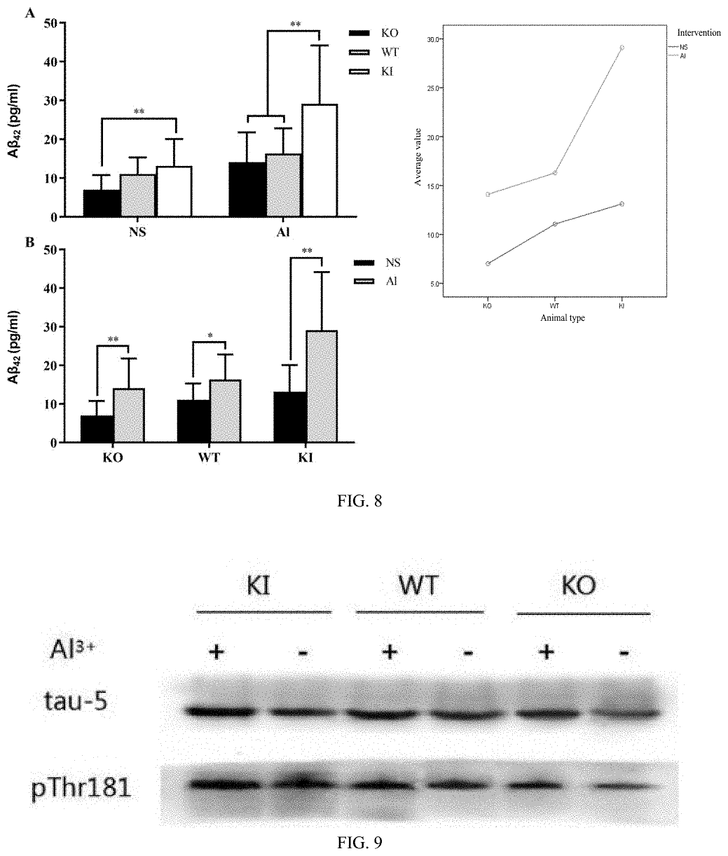

The results showed that there was an interaction between animal types and intervention measures on the Aβ₄₂ content in the hippocampus of mice, and the Aβ₄₂ content in the hippocampus was significantly increased in mice of the KI+Al group (see FIG. 8), indicating that there was accumulation of Aβ inside the brain of mice in the AD model group.

### Example 6

This example was different from the above examples in that the determination of expression levels of Tau proteins in the brain was added. The tau-5 and pThr181 levels in animals of each group were determined by Western blot. The hippocampal tissue protein was extracted, then subjected to electrophoresis, membrane transfer, blocking with 5% skimmed milk powder, incubation with a primary antibody (tau-5 (1:300), pThr181 (1:3,000)) at 4° C. overnight, washing, incubation with HRP-labeled goat anti-rabbit IgG (1:3,000) at 37° C. for 2 h, washing, and developing. The gray value was analyzed with Quantity one software, and the relative expression level of protein was calculated.

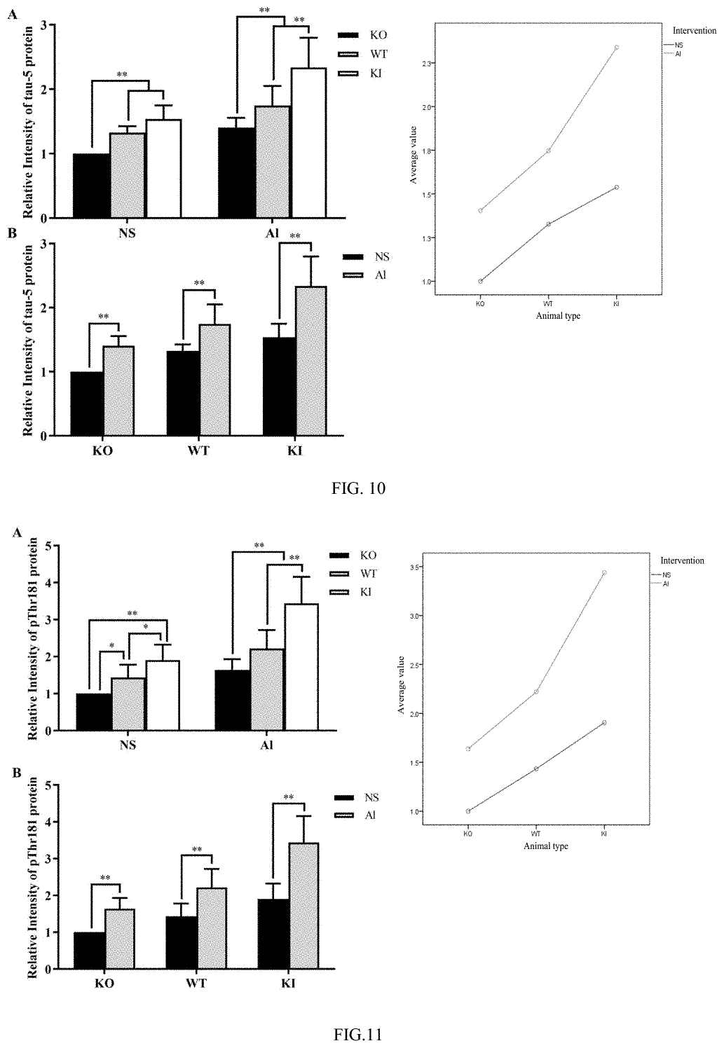

The results showed that there was an interaction between animal types and intervention measures on the expression of tau-5 and pThr181 in the hippocampus of mice, and the expression levels of tau-5 and pThr181 in the hippocampus of mice in the KI+Al group were significantly increased (see FIG. 9, FIG. 10, and FIG. 11), indicating that both the total tau protein expression level and the total phosphorylated tau protein expression level in the brain of mice in the AD model group increased.

Although examples of the present invention have been shown and described above, it should be understood that the above-described examples are illustrative and not restrictive of the present disclosure. Variations, modifications, substitutions, and changes can be made to the above-mentioned examples by those skilled in the art without departing from the spirit and scope of the present disclosure.

Attached gene sequence list

| | |
|---|---|
| <110> | SHANXI MEDICAL UNIVERSITY |
| <120> | METHOD FOR PREPARING ALZHEIMER'S DISEASE (AD) ANIMAL MODEL |
| <130> | GWP202010417 |
| <160> | 1 |
| <170> | PatentIn version 3.5 |
| <210> | 1 |
| <211> | 26220 |
| <212> | DNA |
| <213> | Mus musculus strain C57BL/6J chromosome 11, GRCm38.p6 C57BL/6J |
| <400> | 1 |

| | |
|---|---|
| acaaaggtgg aaaggagatt tagtggagtg gcctcagtgg cctcagtcac agagcagaat | 60 |
| ggaggggagg tctgatcaag gcaggaggac cagatgctag gccaaagagt ttaagtgcac | 120 |
| ctgttgaaag ccggtagaca tctttgagca agagactgcc gtgactgaaa ggactatgtc | 180 |
| tgaaaattga tctgaccagg agcacagctg gcctgaaaga ggagcaggaa gactactaag | 240 |
| ttcctagatg tccagaaccg atgcagaagt cttgaagcag gcatgctgtt tgtggttaga | 300 |
| agaatagcaa ggcaagacct agaaattcag agcctctgtg gctggtgaga tagcaagcgt | 360 |
| tcaattctca gcaaccacat ggtggctcac aaccatctgt aatgggatcc gataccctct | 420 |
| ctggtatgtg tgaagacagc tacagcgtac ttgtatacat aaaataaata agtaattctt | 480 |
| ttaaaaagaa agtaaaaaga aattagcccc atttgcgtca ttccagaata attcagtaat | 540 |
| taagctcata tgggaaaaaa tgtttgtggt gctagaaaac tgaacccaaa gttatctcaa | 600 |
| aattttattt tgggggctgg agaggtggct caggggttaa gagcactgac tgctcttctg | 660 |
| aaggtcctga gttcagatcc cagcaaccac atggtggctc acaaccatcc acccccccag | 720 |
| cccagaagtg tgtttgtttg tttgtttgtt gactcacttt caccctgtaa tacaattcag | 780 |
| gctggcttgg attctgctat atagcccagg ctgatcttga acaagtctca gcctatctga | 840 |
| atgctgggat tacagatgtg agccaacaca ccgagcaggt acaacgttgg agttttagaa | 900 |
| acaactgctg ggatagcagt gcacaccttt aatctcagca ctctagaggc agaggcaggc | 960 |
| aggggatctc tgtgagctcc aagccagcca gggcaacata gtgagaacct gccttgaaaa | 1020 |
| ataaagaaaa aacggcatgc aaatgaaagc ttcaagacaa gttttcgcct ctttggcaag | 1080 |
| aagtaggtgc ctgtctctct tccttgcatt tgaacaggct ccagctgtcc aagagaattg | 1140 |
| agctttgggg aggtaagata ttaagctaaa gccacagccc acgcctacgt agctggaagg | 1200 |
| agaactaatg accaatttca accggaagct tgacatccag caggtggcca tctccagcag | 1260 |
| gcaccttaaa aattcagctc tcacgctgta caaaggtgtt tcaataaacg agataaaaat | 1320 |
| aactctgtga aaatgccttg cagtgtgtgt ggtaagcatc attagaactt acctatttaa | 1380 |
| agatttttct tcgctagggt cacagtcaag atacctaatg ggtttgaatg ctccttgatt | 1440 |
| taaaaacaat tgtttttcca cgaaaacgct aaagtgtatt acggtcaagt taatctgttt | 1500 |
| cgagaagatt tgtcaaggaa atcattcact ttggccatta acaaaaacag gtggaaagaa | 1560 |
| agcaagcgaa cagctggccc tggaaataga aagtgcgctc tatctggagg gccacatttt | 1620 |
| aggagactcg gggagcctgt agcacttggg gttcacggtg taccgcctgg agagccagcg | 1680 |
| agggtgaact gaaaagcgga cgatgaccga cacaccgctc tgcctggacc ccgcggggac | 1740 |
| cgagggaggg gacccctgga ctagacaaag gctgcagccc cgggaggccg ggtgcgcgtg | 1800 |
| cgccacggaa tgcgcgtgcg cgcggcggtc agcaccgcgg cccgaggcgt ggcgggaccc | 1860 |
| ggggagggaa gcgcggagag gggcgagccg agcggcgcgg agagaggagg gttgaggggc | 1920 |
| gctcgcgtgc gccccctagc ggtcggaagg ggaggtgccc gcccaggcct cgctgcccgc | 1980 |
| ccctccccta gggaggttcg cgctcccgtg gctcgcgcct gcgctgcccg ccggcctcca | 2040 |
| gaacgcgctt tctcggccgc gcgcgctctc agtctccgcc acccaccagc tccagcacca | 2100 |
| gcagcagcgc cgccgccacc gcccaccttc tgccgccgcc gccacaacca ccttctcctc | 2160 |
| cgctgtcctc ttctgtcctc gccttctgtc gattatcagg taagcgccgc agctctgaaa | 2220 |
| tccagtcccc tctggccctg tgcgccgtcc tcctgccacc cctcgcccca gagatggatt | 2280 |
| tagggagctg ggcatcaatc aggcgcaacg gcgggggcct ctctgcgccc taggcgctgg | 2340 |
| ggagctggga aggaacgcgt gcaagagatg cacgcacgcg agcgaatgcg tgtatctatg | 2400 |
| agtgctgggg tccccactat cggctccgcg caaatgaggg gggctgtgcc cgcagggtgg | 2460 |
| ggcgcggatg acggtgacct agggaagggg ttgttctgta atctggatct cagccttttg | 2520 |
| ccgcaatggg cgtgggtctg tgagtgtgca tgggggtcca acgacgaccc ccgaaacaga | 2580 |
| acctgaaatc ggatttctct gctcctgatt gccatcagcc ctaaggaagg tgtggatcgg | 2640 |
| gttctagaaa agatgactgc cttcatgtct ctccacctcc ctgacagcga ttctgacacg | 2700 |
| gccttcactg gccctgattt aaggcggtgg cccgccccct ccccttttcc tccttcagaa | 2760 |
| gccggcaggg gacatttggg ggctgggagg aatccggagg atggggaggg gtccaggcgc | 2820 |
| tgtcacgtta gtcgcccttc cccctgcgcg cgcttggtac agtgacaggg gcagcgccgt | 2880 |
| gcccgagaga aggtagtgcg cggatcccaa tgtgggagct cgaagagtca ggggtcacct | 2940 |
| ggcctcgcgg tgtcagcggg ctggctggca gcggctcccc gctcgagggc gcgcagcgtt | 3000 |
| gcagccaggc acagacccac gcgagggctg caggtgcatt tcggggcgcg tttctttctc | 3060 |
| tgcgttctgc gccgggctcc cggcatgtgc gctccctgcc ggaggcgcgg gctggcgcgc | 3120 |
| agggctcgcc cctcactgcg gcagtgggtg tggacctggg tgggcgagga ggggggagta | 3180 |
| cgatgtgtct cctcccgctg ccacccctct ccgttttttc ccccctctct gaacgcgagg | 3240 |
| tgccatcttt ttgcggcgag tcacgtctct atcgtagcat gccaagagct gtggccaggc | 3300 |
| tttggaggac ccggcttgga cccatgttaa agaacagggg gtgatgaaca cggaagcctt | 3360 |
| ggcgttttgc ctctgcagcg gtgcgcacca gagcgttcac ctgaggtcgt ctaggggagc | 3420 |
| tcgctgcttt gttcgtcgtg tgatttagga tctgcgctcc cagccctggt tttttggggg | 3480 |
| agtgtgcggg gggaggcggg aagctttcat tcgacgagga tatttgccta cgcactcgga | 3540 |
| gcagctccat ccttttttcg ccactctttt cagacccttt gttggatcta cagacttgcg | 3600 |
| gcagcccact ccccagttcg acccagagcc acatgggtgg gattcgagat agactctcgt | 3660 |
| gtgcaggagc cctcagagga agcacatccc tccccacacc caggccatcc tgaggaccac | 3720 |
| accccacttg tgtttcactg ctaggggagc cagataaccc tcgggaatgg ggagattcgg | 3780 |
| caagctcaac tccgcatccc caccaccacc accaccgttg aaccctgttg cctctcccgg | 3840 |
| tgggagagtg tgaggagagg gtatgagatc ccctgggata ctgcggccac acccatccct | 3900 |
| cccaacccaa ggagagaggg ttaagggttg ccataggaac cacaccagcc agaggaggaa | 3960 |
| cctctgggcg ttacctcacc ccctacctcc caccccctac ctcccacccc cccccttact | 4020 |
| ccgcggagat ttagaatcct ggggtagtct gatagtgcct ggaagacacg aggaaggggc | 4080 |
| ccctcatccc tgatgcgagc atcattctct tggcactggc aaccatagcc atccagagag | 4140 |
| agagagagag agatgcctct aatgtatttt attataagcg tgctgactga tattatttta | 4200 |
| ttaatatcat ttaaatgagg gactttcatt tttaaaaaaa tccacatcta caacagccct | 4260 |
| tgaaaccaga gagggataga aagctaagag gtcccagcct ccccaccgta cagtaagctc | 4320 |
| ggtcttcgag gttttctggt taattttacc gctgaatact ggtgcgggta agcagttttg | 4380 |
| agaccgatgg atgagcttct taagggccga attgcccacg tcgactgctc actcagattg | 4440 |
| atgtaggctg ggggtggaac gccctctccc gatcaaaccg cggcctgagg ctgtggcatc | 4500 |
| gtggccgggc ccgcctggca gaaatttcgc gagcatctga aagataggtt caaatactac | 4560 |
| ccatccctcg ccacacgacg ggctgctggg aacaggattc ttaagctaga agaagccagg | 4620 |
| ctcttgggct ggatgcggaa aggggaaggc tgggaggggg ggctgactat agcgggcccg | 4680 |
| ttgggcaaag gtgtcacagc ggaaaaatca gcgggagggg gaggagacag aacgttttac | 4740 |
| ccagtgcaga aacaaagctc agatcacagc gtcttcgaac tcccattttg cactgtgttg | 4800 |
| aggggtcgag tgaggtactg tgctggccaa ggcccgtgtt gttaattaaa gatcgtgggg | 4860 |
| gttgctgctg agtttgagag actccattcc tggctctgcc ctttaacaag ccagtttcat | 4920 |
| tcacctcaga agctagaagt ttgtgctgaa gccagagctg tggagcgctg agctaggctc | 4980 |
| atccgaaagg gttcaagagc caagatttca cccaccaacc tccgcgggtg tgtgctccct | 5040 |
| tttatgtccg ggaggcccta tttatcctct agtgttcaag aaattgtaat cccaagtctt | 5100 |
| tttttccggc tggttagcat tgggtgtatg acagggtcag agacaaacta accaggccat | 5160 |
| tttcggctca gtccccgggc tccacaagac tgcccaaaat tttcacggat cctacgccac | 5220 |
| ccattcaact gtcaggtctc atagcagaaa aagaagtatg aagccatggt aggttaattc | 5280 |
| ggtggttaca attctgagat gtcatctggg gttcaatgat actgcttgtg tttgccacat | 5340 |
| aagaattgct tgcccactcg ccatttgcaa agcttctcta aacaaatggc tgtccagata | 5400 |
| atttgtgtgc ccattcctcc cccagctgcc aggccgggtg taatgactgc tgatctgttc | 5460 |
| tggacagttt gcctcaccag cagatgtgta tgatatagta ccctagcttg gctttgcggt | 5520 |
| tcaccttcgg atggtttgaa tatttcttag cttggctggg tggtttgttt gtttttttta | 5580 |
| aagatgaatg acagcccacg gtgtgcctct ttgccctctg cacagaccct gtctgtctac | 5640 |
| acaattgtgt gttggtggaa gggtgtggct tttaagtggc ttctagtgtc aggctccgtt | 5700 |
| ccccagagag gaacaaagga agcagagaga gtgcccagga attgcaagaa gaaaacttgg | 5760 |
| ttggattgag ttaaggtcag gcttgctcgt tgagaacatg agcccttgta cagagcaagc | 5820 |
| tctctggccc actcgtcctt ggagggctgg gtcagcctgg ggatgcgatg gggctggagt | 5880 |
| ggaatttctg cagactgact tctcctcccc tttaaacatc aacgtgtgca gttcatttgt | 5940 |
| tggaaatgtt aaggacttgt gttgcattcc gtctcccctg agcatactgt atatggctct | 6000 |
| tgtgagttcc tggctacgtg tatgaacata cattgctttt tttttttcag ttgaatatat | 6060 |
| gaacctttcc cactatgcct tttcttatat ttatttagtt aagttcttcc ttggtcccct | 6120 |
| cttgaaaatg aatcacttca aaagaatgtt tttagatatc taatataggg gctagatata | 6180 |
| gagtgggtgt ctggcccacc attgtttgac agatccgtgg gtaaaatgga gagatacagg | 6240 |
| tcaggagggc atccaagaga acatagagag gggcaaatat tcaagctcac agcatccggg | 6300 |
| aagctcgggg acaaatccag tctattttgt ctatgtgtcc agttggtgcc cacaccgctg | 6360 |
| tctgtgatct gctggacacc tttgctctgc ctggggcccc ataatgtgca caggtggctc | 6420 |
| tcctgcccct tgttgttgaa ggggaattaa gagcctagag ggggctacct gtgggggtgg | 6480 |
| ccatgttccg gctgcaggtg actaggatgg agatggggag cctcactctc tcatcagcca | 6540 |
| ctgtagtgac actcattgga ttcctctgca agcaggagtt aggggtacct ggccaagtga | 6600 |
| gaaggagaaa agtccctggc tcctaagcag ggcctcataa ggctcctgag acttgggtct | 6660 |
| tttcctggaa aaccaaggtc ctcattgggg ttgctgaagt atctggaaag tggggtgtgt | 6720 |
| tgggggcatc ttgtgggtag aggcgggaaa tgctgctaaa cattatagga cagctgccaa | 6780 |
| atgtcactca ctggctctgg catagagaag ccctgctgta gaaacagtgt tttgctggca | 6840 |
| ctgttatctc aattgtatat agtgcctgtc ttctaggagt gctgcatgct gctggagatg | 6900 |
| atctcatata gttctcactg tgaccctctg acacataggg aagccgaagc ccagagaggt | 6960 |
| ttcataacca actaaggatg cacagcaaag aacacacagc taagccccag gagatttaat | 7020 |
| ggaactttgt gggctagcct gggaagctaa tcgggctttc caaccaccca gttcttttgt | 7080 |
| aagagtctga ttggctcaca ggctggaata agatacggaa gtgtggattt gagtccttgg | 7140 |
| tgtctgcttt ggacatctgg gacaaaggat ttccctccac cttggtactc tgcttatcaa | 7200 |
| acagagagca agtctttccc ggaaacctta acccgacctg actgagcgat tctccactca | 7260 |
| tctgtttcgt gaagggccag ccaggtttga ctggcttaaa cataactgtt aattaagata | 7320 |
| ggggcatcaa gggcagggaa aatggctgtc ccctcgcatg agggccttcc tgagttcaga | 7380 |
| tctctagcgc ttacataaaa gctgagtcct gctgtatgca tttattaaac cattgctggg | 7440 |
| cagggggtga aaacaggtga gtccctgaaa ctcactatcc agaaagctta gccaaattca | 7500 |
| caacctctgg gtctctgaaa agcagggggg gggggaggca cagaggggga ggggggagag | 7560 |
| tgagtgagag agggaggaag atatcagttg gataattgga ttcagctgtg gccagggtga | 7620 |
| aatcactgag gtttgggggg tgtcagattc ctgtcttaga ttctgtccct cccatagctt | 7680 |
| gaacattggc acccatggct gaggcccagc cctgaaactg tggatatatg tggagctgtg | 7740 |
| agcacaagaa gctctcgttg caagcatccc aagtctccgt gggcatcgtg ggccctgcag | 7800 |
| gctatgcttg ctgcctggcc agactccaaa gtacatttgc attatttctc tgtgccacag | 7860 |
| tgccactgag accgcttcag ccatgggctc actgctgctg accgtgagag gtgaggcagc | 7920 |
| cgaggtgagc aggcccctcc tagcccgcca gtgccagcaa tggaacacat actgtcacac | 7980 |
| ttgcttcact actgtcacct tgccaaagca gttgctggca aataattcct tccccgcctc | 8040 |
| cacttgctag acttctgcct gtagggctcc cagggaacat ggggggcagt gttgaggtta | 8100 |
| cccccagcta gctgtggcat tctagattgt tctctaggca tgtggataga atgggcatct | 8160 |
| gcaaaatgaa acctcaaggg acttccaggc gggtatctta acctttcctc ttcgctcatg | 8220 |
| tgccagcagg ctcttcactg agcccaatca gcttctcaat agaaaatcgc aggaagtgta | 8280 |
| tctcttacct gagcaattca gagaagtgtt gtaagcgcct ggagagcagg tgcccagcac | 8340 |
| agagggcatt tagaggtcat ttggtttaga acccttggat gccagtgcca gcttcccaac | 8400 |
| aatccaacct atggtggagc ttagctactt agtaactggc tttttttatt tttaatatat | 8460 |
| tcatatgtgg cctggggtat tatagggcct gtccagaaga ttgcctagga gtttaaatga | 8520 |
| ggaaatgtga tgcttgccat ggcctggcat gtgactaaat ggcattctta accatctgta | 8580 |
| tcctgggcca ctggtgttgg aaaacatgca gcagataaat cgcaccaaga aagaaccaag | 8640 |
| cccagatcct ctttcggaac caggcattgt tgtttgggcg gaagtggatc tccaagatca | 8700 |
| cacacaccca tgtttaagtg tacacacaga actcttgtgt acagggactt atctgctgtc | 8760 |
| ctgcttctgt ctgcttcctt cctgaatcct gctccatgcc aggattagac tgcaaactcc | 8820 |
| ccacaccagc cttccctata cacccactgg cgctctctcc ctctgccccc ccctccctcc | 8880 |
| ctccctcatg gaaagggtga cctggctcag tgtgttaggc ccgaggtctc ctggtttact | 8940 |
| cattgaagca aagtgactac tggtgactga gagcgtaatg acagtatcaa aaggcagtgc | 9000 |
| gggggggggg gggggggtga gattgcaaac gaagtggctc ccctcctaca gaagtcagta | 9060 |
| gcccggtgag tggccttcat ggctgacagt gtcaagagag acaaatgtta agtaatccat | 9120 |
| agtcctgaga aatgaagcct gggaattcca ggcacccaat aaaccgagta gagtgtgagc | 9180 |
| caatagaaca agccacagaa gcctcctcaa tgtggaatca aggccaaggg cgtaatagag | 9240 |
| cagttttgga tcatttttag gaatcatttt tccgcttcat ttaattattt atttgtgtgt | 9300 |
| gcgtgtgtgc atgtgtgcat agatgagtat gtaagggtgc cctcagaggt caaaacagca | 9360 |
| tcaggtctga cctcctggag ctggagttac aggtggttat aagccatgga tgtgaggacc | 9420 |
| aaacctgagt cttctacaag agcaatacat ggtcttaacc acttaccatc tctctatccc | 9480 |
| cttgaacctt taaaatacag tgtgtgtgtg tgtgtgtgtg tgtgtgtgtg tgtgtgtgtg | 9540 |
| tgtgtgttgt gtgtttattt actgttgtat taattatcta atctcactca ggggtttcta | 9600 |
| cctgtattag tcagggttct ctagagtcac agaacttatg gataatttct aaatagtaaa | 9660 |
| ggaatttatt gatgacttac agttggcagc ccaattccca acaatggttc agtcgcagct | 9720 |
| gtgaatggaa gtccaaggat ctagcagtta ctcagtctca cgcagcaagc aggcgaagga | 9780 |
| gcaagagcaa gagctagact cccttcttcc aatgtcctta tattgtctcc agcagaaggt | 9840 |
| ggagcccaga ttaaaggtgt gttccaccac acctttaatc ccagatgaaa ggcgtagccc | 9900 |
| agattaaagg tgtgttcctt aaactcggag attcaatctt ctggaatcca tagccactat | 9960 |
| ggctcaagat ctccatacca agatccagat aaggatctcc aagcctccag ataagggtca | 10020 |
| ctggtgagcc ttccaattcc gaattgtagt tcattccaaa tattgtcaag ttgacaacca | 10080 |
| ggaatagcca ctacaatcca ccccttgtca acttgacaca aataatatct catgttcaca | 10140 |
| tgaaacaata acaaggttgt aaatacgcct aacatgatat aactatccct cgtacaatcg | 10200 |
| caaacgcatt agtaaattta caatgggcat tcatattact ttataatcct cgtttctgca | 10260 |
| actggttacg tggccttaat tggtatttat aactaccttc ctctactacc cattctgtat | 10320 |
| ttccttcacc ttcagccagc acctcagcag gtcttggctc ttttcctgga ggattgaccc | 10380 |
| ataccttcat tcctgatggg tctgcgtcct ttgtcatcct gcttggatta ggctgttgta | 10440 |
| gtttcccatt gactttaatc acaggacatg gtagtactaa gagacgccct aagggatctc | 10500 |
| ctgcactcca gacataatct tgcttaccac cattgtgaag aggtaatcca atttccccat | 10560 |
| ggtaatctgg atctatcacc cctcctaaca ctgttattcc ttttttagcc tgttggttta | 10620 |
| agggcattag aagcccaaaa tgaccagggg gaagtctgag cttccagttc aatgaaatgt | 10680 |
| ttgttgtagc tcctggtagg agcactcccc tctctggagc caaaacttct aagccagcag | 10740 |
| aacctagagt tatggggaca ggaagcaaaa attttcctag agggtcacta ggagtgatag | 10800 |
| taagtggaac tattccgttt tccacccctt gattcctgga cccatgaatc ctggctatgg | 10860 |
| gtgaaactgt accatatatt gagcgctgat tcaaagcata tactgccttc tgaagaactc | 10920 |
| tgccccagcc tttcaagctg ttaccaccta attggcgctg taactgcgtc ttcaaaaggc | 10980 |
| cattccatct ttctatcagc ccagctgctt caggatgatg gggaatgtgg taagaccagt | 11040 |
| gaattccatg atcgtgagcc cactgtcgta cttctctggc tgtgaaatga gttccttggt | 11100 |
| cagaagcaat actgtgtgga ataccatgac gatagatgag gcattctgtc agtccgtgaa | 11160 |
| tggtggtttt agcagaggca ttacgtgcag gaaaggcaaa tccataacca gaataagtat | 11220 |
| ctactccagt aagaacaaaa cgctgtcctt tccacgaagg aagtggtcca atgtagtcaa | 11280 |
| cctgccacca ggttgctggc tggtcacctc gaggaatggt gccatatctg gggctcagtg | 11340 |
| ttggtttctg ctgttggcag atctggcaat cagcagcagc tgtagccagg tcagccttgg | 11400 |
| tgagtggaag cccatgttgc tgagcccaag cataacctcc atctcgacca ccatggccac | 11460 |
| tttgttcatg tgcccattga gcaatgacag ggatggctgg ggagagaggc tgattgtcca | 11520 |
| cagaacgggt catcttatcc acttgattat tgaactcctc ctcagctgaa gtcacctttt | 11580 |
| ggtgagcatt tacatgggac acaaatatct tcacatcctt tgcccatttg gagagatcta | 11640 |
| tccacatact tcttccccag atgtctttct caccaatttt ccaattgtga tctttccaag | 11700 |
| tccctgacca tccagccaat ccattggcta cagcccatga gtcagtgtat aatcgtacat | 11760 |
| ctggccattt cttcttgcaa acaaactgta ataccatgtg tactgcccga agttctgccc | 11820 |
| actgtgaaga tttcccttca cctgtgtctt tcaaggttgt cccagaaagg ggttgtaatg | 11880 |
| ctgcagctgt ccacttctgg gtggtgcctg cataacgtgc agagccatca gtaaaccagg | 11940 |
| ctctagtctt ctcctcttcg gtcagttgat catagggaac accccatgag gctataggtg | 12000 |
| catgcttggc agcagatggc attgtaacag gagtagaaac cataggcatt tgagcaactt | 12060 |
| cttcatgtaa cttgcttgtg ccttcaggac ctgctctggc ccgatcacgt atataccact | 12120 |
| tccatttgat aatagactgc tgctgtgcac gtcccacttt atgacttgca gggtctgata | 12180 |
| gtacccagct catgatgggt agttcaggtc gcatagtgac ttggtgtcct attgtcagac | 12240 |
| gttcagtttc cactaaggcc caatagcagg ccaagagctg tttttcaaag ggagaatagt | 12300 |
| tgtctgcaga tgatggtaga gctttgctcc aaaatcccaa aggccttttc tgtgattcac | 12360 |
| ctacaggggc ctgccagagg ctccaaacag catctctatc agccacagac acctcaagta | 12420 |
| ccatcggatc tgctgggtca tatggtccaa gtggtagagc agccagccag taaaatcatt | 12480 |
| tttcgatttt ttccccatct tgtagaaagc tttgtgcact gaatcaccta attcattaag | 12540 |
| aaaatcaagg gcattagctt ctttaagttc ggaatatagt ttcaaccatg ggtcttcaaa | 12600 |
| attctctgag ctcccaggag ggagagaatc tggagaggtt tcaatatttg aaagtgctgg | 12660 |
| tggatcaaca agccaattcc agtattttaa aagattcatc cttgtacttc tgttactcta | 12720 |
| gaaccactcc tggtaccaac ttctgtatta gtcagggttc tctagagtca cagaacttat | 12780 |
| ggataatttc taaatagtaa aggaatttat tgatgactta cagttggcag cccaattccc | 12840 |
| aacaatggtt cagtcgcagc tgtgaatgga agtccaagga tctagcagtt actcagtctc | 12900 |
| acgcagcaag caggcgaagg agcaagagca agagctagac tcccttcttc caatgtcctt | 12960 |
| atattgtctc cagcagaagg tggagcccag attaaaggtg tgttccacca cacctttaat | 13020 |
| cccagatgaa aggcgtagcc cagattaaag gtgtgttcct taaactcgga gattcaatct | 13080 |
| tctggaatcc atagccacta tggctcaaga tctccatacc aagatccaga taaggatctc | 13140 |
| caagcctcca gataagggtc actggtgagc cttccaattc cgaattgtag ttcattccaa | 13200 |
| atattgtcaa gttgacaacc aggaatagcc actacactac cctaccttag atcattcagt | 13260 |
| tcccaggtaa acaacccaaa gcttttatat ttataataag ccttaatcag tactaaagct | 13320 |
| gggcagatat cttctctcga ctattcctat ctacttcgct agccataacc ctgaattcta | 13380 |
| acttgccatg ttccgtctga gctgctgctc ttaatttcaa ttggccagcc ctcatggcca | 13440 |
| tgttttcatg actcacctac cctatggcat ctcctcttct ctccaccttc tttcctcaag | 13500 |
| gtccctgcct cagaccccag cctgggaacc aaaactccac ctatctctct cttctgccga | 13560 |
| gcagtaggct gtagggataa tttgtggggc ggggctacat agcatcacct gggtctgctc | 13620 |
| gaggtcctcc tccttcctgg gggcaaccag ggccagtatt tagcattaca atagcaacag | 13680 |
| accaaacctc aacactttgt gtgtgtgtgt gtgtgtccac atgtgggctc acagaacaat | 13740 |
| ttgtgagggt cagttctctc cttccaacgt ctgagtcccg gacacaaact cgggtcatct | 13800 |
| agtttaggac caagcacttg tttggccatc ccactggacc tatttatacc ttttggggga | 13860 |
| gaaaagtctt atttgtgtgt gcatgtatgt gcgtgcttct atagtgtatg tgtatgcatg | 13920 |
| gtatgtgcat gtgtacgtga gatggaagcc aggagacaaa acatcagatt ctccatacat | 13980 |
| tgccatccac cttatttgtt tatttattta ttaaaatttt tactttatta ttttatttta | 14040 |
| tatgtatgtg tgtttggttt gcctatgtgt gtgcaccatg tatatgcccg gtgcccccca | 14100 |
| cagaggccag aagagggtgc tggatcctct gggactggac ttatagatgt ttgtgaactg | 14160 |
| ccatgttgct actggaaatc aaacccaggt cttcagaaga gcagccagtg ctcttaacca | 14220 |
| ctgagccatc tctccagccc cctattagtg tttgaggcaa catctcttgt tgaatccaga | 14280 |
| gctaagtaat tgactagaat ggctggttca tgacatctgg gatcccctgt ccttgcttga | 14340 |
| gttcaccatg ctggtgttac agatgagcat cattggtgtt gcgaatctaa aataagagtt | 14400 |
| tctagggctt ggtgggaggt ggcttgggag gtgaagtgtt tgccacataa acataaagac | 14460 |
| ctgaattcaa atccccagcg tacacataca agccagatga ggcagtgtgg ttcctcgtgc | 14520 |
| tttcaagact ctccccagcc tctagcttgt acttatagaa gtaatgcaat cgttatagca | 14580 |
| aagatataat agccacgtga tggttccagt tacctgatta gtatagcaaa taggatagac | 14640 |
| agagaaggct tcagaacaag cccagatgac cctgagcagg agcttggttg tggcaggagg | 14700 |
| ttggcctggc tgcagacagg ctctgtggat cccagcctta agaaggtctt tcatttctcc | 14760 |
| tacaaaaggc cagaaaagca cctaccaggg agacatttgt gttaagttaa aggcaggtat | 14820 |
| actcctctct gcccactctt gcctgtcttt aaggccccca gaatacacat cagacacaac | 14880 |
| tttaaactcc tcttaggaaa aagttttatt caaggtccaa caatagaaag aaagtttgaa | 14940 |
| atccacaaga aatgaatccg tagtcactga taatggtggc acttcctctg tggcccttcc | 15000 |
| actccctgcc agcgaggagg agagggacat ctgtgatctc tggtccctgg tcttccttct | 15060 |
| ccttccagcc tgtggctcag tgttgttttc ccagaaagag tgggtgatgc tgtcctggta | 15120 |
| ccctcgccct agcggtgaca cagaagatcc aggcatgtac tttgatcttg atgtcttcct | 15180 |
| ccctgggaca caaaggatgt cagaaaacct ctgtgtccat aactgcctgt caattatgtc | 15240 |
| aattagtctg cagccagcat aaagtgataa gcagggagcg ctgggaagac tcacagaggg | 15300 |
| tgccttctcc aggacagctc ttccaccaga aggcttcaaa ctatgtgtaa acaaaattca | 15360 |
| agggggctta ccaagcaaaa acaaatgaag caaaacaaac taacaaaaat tcttaaaatt | 15420 |
| gttctctgtc aaatctagcc ctgccattca tttacactgc aattcaggag gtgtgtgtgt | 15480 |
| ctgtgtctgt gtctgtgtgt gtgtctgtgt gtctgtatgt atgtctctgt gtgtctgtgt | 15540 |
| gtatgtctgt gtgtatgtat gtctctttgt gtatgtctgt atgtctgtgt atgcatgtgt | 15600 |
| gtgtatctgt ctgtgtgtgt gtctgtgtgt gtctgtctct atgtctgtgt ctctatgtgt | 15660 |
| gtgtatgtct gtgtgtgtgt gcgtgtatgt gtctgtgttg tatggcatat gctttcacat | 15720 |
| atgtgagtgt gtggaggcca ctggctgagg tgtggtgtca cccactatgg ccccacacct | 15780 |
| tatttttaat ttttatttta ttattttata tgtagaagtg tttgcctgca tgtatgtttg | 15840 |
| tatgccatgt gtgtccctgg tgcctgtaga tgtcaaaaga gacatcagat tacctgagag | 15900 |
| ttacagatgg ttttgagctg ccctgtgggc tctgggagtt gaaccaggtc ctatgaagag | 15960 |
| caggcagagc tctttaccta ccaagtttcc tcaccagccc ctccacacct tactgagctc | 16020 |
| aacattttca ttagaggagt gactagccaa caggatccat ctgtctgtct gtctgtctgt | 16080 |
| ctgtctgtct gtctgtctct ctctctctct ctcacacaca cacacacaca cacacacaca | 16140 |
| cacacacaca cacacacacc gacccatcca gtgtttcaga catatgctac cacagctggt | 16200 |
| ttttatgtgg gcacagggat tgaactttgc tcttcatgta tgagaggcag gcactttagc | 16260 |
| cactgagcca cccctccccc actgagcctg aggaacattt tttttttttt tggtttttcg | 16320 |
| agacagggtt tctctgtgta gccctgactg tcctggaact cactctgtag accaggctgg | 16380 |
| cctcgaactc agaaatccac ctgcctctgc ctcccgattg tgagtgctgg gattaaaggc | 16440 |
| gtacgccacc acacctggcc tgaggaacac ttatatgtcc cccaaattac ttgtcttacc | 16500 |
| ttatccttcc ccggtggcct taccccccaa aagaggtttg ccgatgagct aataatctga | 16560 |
| gaccagtcct aagagaaggt tggacttaaa gactgacttt ggttacacag ccataaatgt | 16620 |
| gaatattgac tattgtatgt ctggggtttt tatgttgagc ttgctgaaaa cctggtcact | 16680 |
| ttttaacttt gttattggca gtgcatcaga atttaagtaa atttatgtta attgtcagag | 16740 |
| gaaaagttgg cactgtgagt aagacagagt aaaccacagg atgcctctag ggcacaatga | 16800 |
| aaacccacag cgtggacaaa taggaacagg tctctttcaa agctgcccat tgcctggaag | 16860 |
| actgagacag catttggagg tctcaccagc ctacttcctg cctagtcttt ttccagaaga | 16920 |
| acacaggaaa tactacatgg cttgaatctg ctgtgactac tgtggggtca ggctctgccc | 16980 |
| cgcccctatg cagccagctc aggaggatag actttgaaaa agaaaataaa aatccctaaa | 17040 |
| tccgcttaaa cgcttaaacg tggaagatct tcacactccc caggaccttc tttaagctgg | 17100 |
| ttgaatattc tcctccaata aacctcttgg gaatgatatg cctcccaggt ggctcagtgc | 17160 |
| tggggtgagc ccctcctccc ggctccaaag tccagccctt cctcctcctc cctcctcctt | 17220 |
| cctccctcct ccctgtgcct gagaaaaggc tagtcatgga tgcactgttc cctgcagagt | 17280 |
| gctaataagg gctgagtgca ttaaaatata tgccttgctt tttttttttg gtatcataaa | 17340 |
| atattattaa caatcacaag gcagccctta atggctgttg gagaatttca gaaaaacatt | 17400 |
| tctttggata agcaggattt gtgccataag ttgacagtcg ggaccaaggc ttagaataag | 17460 |
| atttaagact tgatccatca tttgttgctg acaaaactga actacagaag ccagtgagct | 17520 |
| gctcacggtg tggtcttggt catcacccac tgctgcatcc tgaatttaac tgccagtttc | 17580 |
| ccgttggcca ataagagatt tttgacaaag atggagaatt cctagacctc tgtccttcgt | 17640 |
| ctgcaaaatg tgaatgatac agtctcccct tggaatggag tggatggtaa ggcctcagcc | 17700 |
| ttgatgtcta ctgagtagtc acagaactgg aatgaaacag tccatgagaa atccttgggg | 17760 |
| tagctcagaa gtcagtgtcc cactggaact gctacagctc cctgatcaga ggtcccaagt | 17820 |
| gggctgagtg tagcctgaag tggcacagct gctccctgcc accttgctcc tgtttccagc | 17880 |
| cagccggagg cagctggtcc tctgtagtga acactaggtc agcacgggtc catgtccaga | 17940 |
| aggataaaca ggcggccaca gcccagtctg tccccagtgt agagaggagg agaccccaag | 18000 |
| tcagggtgca ggacagtggg aggggtgacc tcaagtgatt tcgagacctc tccggaaagg | 18060 |
| agctgggact tcctcttggt ctccagttgt gacccttgag tggaatttcc tgggagtgtg | 18120 |
| cccatttttt tatggaactt tcagtgtctt cctaattggt acagacttga cacagaacct | 18180 |
| cacattctaa attttcccag ggtggattct ccctgcctgt ccatggtagc atatgacaga | 18240 |
| aatccaccaa ggtgagagca aattcgagcc agaccttgac tttgatggca tgatactttt | 18300 |
| gtggctcatt aactgaatgg ctttaattga gccgcagctg ggctggagtt cattgactta | 18360 |
| gccctgtgaa cggaaggttt ctttggcctc gtcctccaac ccccagcact ggggattgaa | 18420 |
| ctcagggctt catgagttct atcacagagc tacatctgct gcctgaatgg aaggttcctg | 18480 |
| aagaatgagg ctggtgggaa atccagtgtg caaccccatc ctgtcagggg cattggctgc | 18540 |
| acctcagtga gctccagtgt ctactgaccc cacgtcactg ctccttagac cctcaccatc | 18600 |
| tgatcttccc gcttgggctg ttgaaacctt ccatcttccc atcccccgag tggtgaaatg | 18660 |
| cagtcttcag ctctccctcc atatcctgga gtggacctgt cggtttcccg cagacattgc | 18720 |
| ccacccagct ttgtttctcc agctctgctt ttactgactc caaaatgagt tcttacaggg | 18780 |
| ctggagagat ggctcagtgg ttaagagcac tgattgctct tccagaggcc ctgagttcaa | 18840 |
| atcccagcaa ccacatggtg gctcacaacc atctgtaatg ggatctgata ccctcttcta | 18900 |
| atgtatctga agacagctac aatgtactca tatacataaa ataaaatctt aaaaaaaaaa | 18960 |
| agagttctta ccctggcatc gttatagaaa taattataat tcctagagtg aattctaaag | 19020 |
| ttatactcga tgcagtttgt atcccattgc caaacacttc ccagagcaaa attctgtctg | 19080 |
| tcatgttcat gtccacccac aaatcccttg tctgtcacct gctaagccga tcctgccctc | 19140 |
| acgccccgcc cagaccctgg atttcttctc actgccagcc agctgccatg tcttcttcat | 19200 |
| agatggcacc ttcttaattc cctcatcttc catggcattg tgcaggtggt accctcctca | 19260 |
| cctgtacaga ctccgcagtc tctgtcccct ctgagttgtc cccacctgtg tgcactagat | 19320 |
| ccaggagccc cacacacttc ctctcccatg cattcccact ggagtagctt atgtctagct | 19380 |
| gtcatcctct ttcttgtctt gctttgtgtg catttcctgc ctcctccagg ttatctgtcc | 19440 |
| tctcagagca gacactgtgc atcaccaggc actcctggac ctctggggca taccacagtc | 19500 |
| acccaacccg cactcagagc cccgaacgtt ctggttttgt ttgggttttt gttttgttgt | 19560 |
| tttgttttaa gacagggtct cactctggag cctaggctga cctgaatctc actggatagt | 19620 |
| ctaaggaact ttaaacttgt ggcaaacctc ctgcctcagc ctcctgggtg ctgggattac | 19680 |
| aggatgtgcc tgctagcaca ctctgggtga atatggctat gcaggctgat gctgggtgag | 19740 |
| cctgacctgt ttaaagaaga cgagcctttc cctgctgatc cacagtccca ctgctctgag | 19800 |
| cacctaaaaa taaaaatcac attttcagtg agatacttga ttgcagtctg ttctactttg | 19860 |
| aaccagccaa tcaaccagtc tcccccgtga tccagatccg ttatctgtat gaagacgcac | 19920 |
| agaacacagc aactaccggt tgcatttatt ttactaaaaa ctgtgccttg gtccccgttt | 19980 |
| ccccacgaga ctgggtcatt ttgcaaagga gcaaagaaat ttgggggctt aagaaagagc | 20040 |
| caaatggtgt cttaaaccca ggtctactcc atgtcaggcc cttgggtctc ctccccaagg | 20100 |
| ctccatgctc tctgccagcg ccgcgaggag ccagaggacc tgtctgtaag aatctgggaa | 20160 |
| gcatgcccac gtgaacgggt cacttgtaac gtgagcccgg agaaattatt ttaacctgaa | 20220 |
| agcaagcctc taaaccaggc tgctgcttca tgtggcccat gacggcaaaa ccaaatttag | 20280 |
| catcacagac aggtccaccc tgaacaaact ccactctctt aaaaaagtct tccagctcac | 20340 |
| agaaagcatg ggcaaacatt cacacccacc acagtgccag gaggttccca gtgatcagag | 20400 |
| gctgggggat gagcgcgtaa atgaactcag ggaagagaca gacagtgttt ccaggaggag | 20460 |
| agtcagttaa aggggacagg ttccctggat gcgacaaagg catctttgct gctgggggaa | 20520 |
| aaggtgggtt gatgggacag agggagaccc cgtggtcctt ctcttttaaa ggtcctgagt | 20580 |
| tcgattccca gcaaccacat ggtgatacat aaccaactgt aatggggtct ggtgcactct | 20640 |
| tctggcgcat acagatagag cactcatata tatattaaat aaataaaaat aaaatttaaa | 20700 |
| aaatgtacca taaccaaaag gcacagttcc tggagaccac agtcccactt agtgggtcct | 20760 |
| gtggcgctgc agaatgggtg tctgtgccag cccttatgtg tgcggcagca ggtacaaggg | 20820 |
| tgtttgttac aatggtccac tagataaccc acagaaggct acgcacattc gtgtttgggg | 20880 |
| acctcattgc cgaaggtctt cacggtgact ccaacaatga ggtcccactt gctagtagga | 20940 |
| caaaccctgc ccccataggg ggccccaaac tttcaaagaa cacttgagaa aaacctttct | 21000 |
| ggatttacta aaatttggtg tgatttggga tgaatttaga gaagacgagt cattttaagt | 21060 |
| tgtgggtgtt tgagatcagt gactgacatg ggtcacctaa agtaggccca ggagtagggc | 21120 |
| agtagtggtg cacaccttta attccagcac tcaggaggca gaggcaagca gatttctctg | 21180 |
| agttcagagc cagccagagc aacagagtga gaacctgtct caaaaacaga agggaggctt | 21240 |
| cggaggagtg ggcggagcct gcgggactca acagaagaca ccaaaagaag aaagagccag | 21300 |
| gtctttctct gtgtagccct ggctgtcctg gaactcactc tgtagaccag gctggccttg | 21360 |
| aactcagaaa ttcacctgcc tctgcctttc aagtgctgga attaaaggcg tgtgccacca | 21420 |
| ccgcccggcc taagagccac atcttttttt tttttttttt tttttactgt atataattat | 21480 |
| ttatttttaa tttaagtcaa ataaattatt tgagggattt tgattccttt gctgtccatc | 21540 |
| caggtcacct gaaagagaag acaatgagag atgtgttaat gagttaagtg attcagggaa | 21600 |
| atgtatgtta tcctctcaga aaaacacagc actatcaaat cagtgcagga ttttctctgt | 21660 |
| gaagaacatc aatctttggt cacagcgtta agagctgtgg gttttatagc ccaaaaagcc | 21720 |
| ctctttgaaa tgacctaaga gccacgtctt acgtagcaca gttttaggtt ttactttaaa | 21780 |
| gaagaaagca atctgtgtct ttacttatgg tacaaactct cagatctctg accaatccag | 21840 |
| ccggccaggc agccaggcag gaaccatttc ataacataag ttgtgcttgg gtgtgggtgt | 21900 |
| aatttgtggg tgcagtgttc ccatagtgtt gcagtggagg ataggtgaga aaaatcccac | 21960 |
| tgtccaggca gccccgagga gctgaagagg agcaggcgga gactgcgagg ctcatcaggc | 22020 |
| tgccctggga agcaagtgac tgctggggct ggggtggtcc aaggaagctg aggtcaaggc | 22080 |
| ccacactcac ccatatacat ctccaaagat ccagaagaca tggaagggga aagagccacg | 22140 |
| tcatcaggtt aacgaagcca gttaagtaac tcgtcttcag acgcaagtta tttttcccct | 22200 |
| gaaactaaag tctaaaaata ggtaaattaa tgagtgtctg tctatttcaa accaagttga | 22260 |
| tttctcccct ttccctctgc tatacccatt gtgtgtaatt gcagtgatag catctcatac | 22320 |
| cacggtttta aaccaccctg gggaacaggc tccttagcta acccagtatg ttgaagggac | 22380 |
| tttcacccat gtgtccccag cctctggcat tctgcagggc acagaacaag agctaggcac | 22440 |
| catggaatgg atgaataaat agataacgga tccatgaatg aacgtagtat gagttaaagc | 22500 |
| ggcacacagg ggaaggacaa tgtactttgg tctgtgtgtt cgtctctcta ccttctggag | 22560 |
| ttctgcattt ggatttggca tgctcacagt agaaacatgt gcacagtgtg tgtgtgtgtg | 22620 |
| tgtgtgtgtg tgtgtgtgtg tgttacaact gcctttgggg atttcttgtt tgtttttcaa | 22680 |
| gacagggtct ctctgtgtag ctgtggctgt cctggaattt gctctgtaca ccgagcttgg | 22740 |
| ctatagttgt ctgtttaaaa gttcagtcag gtgcctttaa gttcccctga atcctggaat | 22800 |
| ggcatcatga gccataagtc ccacatcagt ggctgtgaac agggtcgggt ctgtggcctg | 22860 |
| ggttactggc attccagctg actcctagag tttcagttct gcatagaggt gttagattgt | 22920 |
| cagatttggg ttggagagat ggctcagcag ttaagagcac tggatgagct tctaaagggc | 22980 |
| ctcggtttca ttcccagcac tcacacaatg gcttaccact gtctgtgact tagtcccaga | 23040 |
| ggatacagtg ccttctgctc acctccaaag gcatggggca tacacatggt acacacaggc | 23100 |
| acatgtaagc aagacaccca tatcttaaat aaataaataa ataaataaat aaataaataa | 23160 |
| ataattttta agcagctgga attgccaaaa tagaacacag ggtgtccaag agctggagag | 23220 |
| atggctcatt ggttaaatga tccagaggat ccaagttcaa tgcccagctc ccatatcctg | 23280 |
| taacttcaac tccaggggaa ttctcttctg gcctctatgg ccctgcactc ctgtgcacat | 23340 |
| accctactcc ctcacataca tacccataac taaaaatata atctatgaaa aaacacacag | 23400 |
| ggtgtccagt tgaatttgga taccaaaata ataataataa taataataat aataataaag | 23460 |
| aggctacaaa atgtcccctt tagatttcca gaggagcctt caagggaagt ggatcaaaat | 23520 |
| gcttctatca tttcctgtca caccacacac acactgaaaa gccctggact ggaggccact | 23580 |
| gttaaagact gacagccatt ttctcatgtg aagaggcctt ccaagccaag gtgagcacag | 23640 |
| gccagctcag taaaggtttc tttctgtcat tgtctttcac acccgatctg ttccccacac | 23700 |
| tcaaaggatc cactgttctg gacacataag ggctctggga tcatccagaa taggccatgg | 23760 |
| tgctaagacc ccaggattac tctccagata ccaagggctt atagaagaga aaatgtacct | 23820 |
| ggtggaagaa agagcaggaa aggagaatga acacgaaaag ctatttggtc tcacctataa | 23880 |
| taggtggctg gtccacaagg tgagatgtcc tcactgttca ccagacagca gaaatccaga | 23940 |
| atggtgtggg cggtgactgc agcttattaa ccaggctgca gcctttgaca gagcctcaga | 24000 |
| gtcggagagg aactgcctaa gagagtggtc cttaaagtag tctttattct ccaccccacc | 24060 |
| ccaccccgtg tatgacctgt taaaaagata aatatgtaca ccaatgcacc agctcacctc | 24120 |
| agaggatata tctgttcgaa gcatctagct ggattgattg cttggtgggt cacatctgta | 24180 |
| attatagact tgaggtgcta agtaaggatt ataaattcag aatcaacccg ggaaacacaa | 24240 |
| aaagaccctg tcccagataa cactcttaac tccccacaaa agctcaaggc cttctacaaa | 24300 |
| gactgttctt ttaaacacac acccctcccc gccagtggca tgcctgggaa gaggttctga | 24360 |
| tctgccctaa ccatcttatg tcacacagaa acagggaagc ggttgcacca ggcccctgcc | 24420 |
| agctatcctc aacacttcac tgccttacac agaaccttcc agttaagcca caggatgaga | 24480 |
| ctagccgagg agcagccagg catgcttgta cacaccttta atcccagcca cttgggaggc | 24540 |
| agaggcaggc ggatctctgt gaatccgagg ccagcctggt ctatagagtg aattccaaga | 24600 |
| cagctagggc taccaagtag agagagtctg tctcaaaaca acaacaaaat agcttagaag | 24660 |
| catggcatac atgggtatgt acatgtgtct gtgtaaaaca tgtttataac ataagacata | 24720 |
| gttgaacccg ggaaccaaat gttcttgtag acccacagat ggacgggata ggtgagcaag | 24780 |
| gagaaatggg ttagtgtgtt taagtcccta ccttaacata gatgggaacg aacgatctac | 24840 |
| actgcccagt ggggaactgc atgtcatgtg acaacataga tggaactatc tgatttataa | 24900 |
| tcccaaatcc tttttatgtc aaaaatagcc ccactgctat tttttcctct tggtgctttc | 24960 |
| acatagatga cttcattgcg tgcccctcca ggccgttcca gagccagtgg cacttcctgt | 25020 |
| ttggaagggg aggaaatggg tgctgcctgt ctccatctcc ctaccttcct gggtgcagat | 25080 |
| gccacaccct gcccctgcac agtcctcccc caggcagctc atcatggtga gcgggactcc | 25140 |
| ttcagaatcc ctgcctgctc acatcacccc tgcttcctct gaaggactca gagaacatac | 25200 |
| tggagaccct gcggagttaa aggagccgag gcgatgtggt acctcagtgt tctccaagtc | 25260 |
| tttagcaagt acacgcggtt accagccttg gcaggctagc tgagcacctg gcagcctcag | 25320 |
| ccgggagaac cttccagatg ggctgtaggc ttccagcaca ggtgccgaga atgatgctct | 25380 |
| tcccaggctc agacccttgg gctggtcacc catgtggtcc aggtcccagg tcttgggccc | 25440 |
| atttttggaa tttctatttg gagatgtggg ggtggccact gagcatggga cagatggtgc | 25500 |
| attcctggtt cagctcattg cctcagcaca atgagatcgt gccagcatgt ttgcagatgc | 25560 |
| tttctggttt tctccttaag gaagtcgagc tccaaagtgg cttgatagct ttcccatggg | 25620 |
| cccttagctc atagcagcct cactgggcag tgcaggggct gcttggcctt tttcctgtat | 25680 |
| tgcttcctga gagctctcct cccatccttg ctctggatgc tctggtcccc caacccagca | 25740 |
| cccatttacc agaggcctag cattgccctt tgacccttcc cagtcctcca aggaatccag | 25800 |
| actgtagcct tccagtagga atagggtggc tgtgtggatg ccttcctgtg cattggaatc | 25860 |
| ccacaaacac accacacaaa cccatcatct gcaggctgca gaggacacgt atggagtcca | 25920 |
| caagacacgg aggcacatct gcagagccct gctgtgtggc ccaggtgaga gaagcaggag | 25980 |
| atggaagagt tctagacact gactggtgtg cagctgaggc ctgcctggcc ctcagcgctg | 26040 |
| tccagcacca cctaacaaca tgcatggatg taaacttcac acaggattgc catatagcgg | 26100 |
| gacagagaca cagctcaaca tgaatccaag gaaatctgaa tgaggacatt tcttactctg | 26160 |
| tttccctcca taggctttta ggtgaaactt agctggtaat atccctttat ttgtcctaat | 26220 |

What is claimed is:

1. A method for preparing an Alzheimer's disease (AD) animal model, comprising: selecting healthy male human ApoE4 transgenic mice, and intraperitoneally injecting a Al(mal)₃ solution to obtain the AD animal model.

2. The method according to claim 1, wherein, the human ApoE4 transgenic mice express human apolipoprotein E4 subtype but not endogenous mouse ApoE under the control of a human glial fibrillary acidic protein (GFAP) promoter, with insertion of human ApoE4 gene into mouse chromosome 15.

3. The method according to claim 1, wherein, the human ApoE4 transgenic mice are healthy male mice aged 4 to 5 weeks.

4. The method according to claim 2, wherein, the human ApoE4 transgenic mice are healthy male mice aged 4 to 5 weeks.

5. The method according to claim 1, wherein, an 8 mmol/L aluminum chloride solution and a 24 mmol/L maltol solution are prepared with sterilized triple distilled water, then the two solutions are mixed in a volume ratio of 1:1 to obtain a Al(mal)₃ solution with a final concentration of 4 mmol/L, and pH is adjusted to 7.4 with 10% NaOH.

6. The method according to claim 1, wherein, the 4 mmol/L Al(mal)₃ solution is intraperitoneally injected at a volume of 10 ml/kg, with aluminum exposure for 60 days, and interval time of 2 days for every 5 days.

7. The method according to claim 1, wherein, the steps also comprise evaluation of the AD animal model, comprising a Morris water maze experiment and a new object recognition task.

8. The method according to claim 7, wherein, the evaluation of the AD animal model also comprises determination of aluminum content, Aβ₄₂ content, and expression levels of Tau proteins and phosphorylated Tau proteins in the brain.
